(19) United States
(12) Patent Application Publication
(10) Pub. No.: US 2004/0147901 A1
(43) Pub. Date: Jul. 29, 2004

(54) Intradermal delivery device, and method of intradermal delivery

(75) Inventors: Daniel Py, Stamford, CT (US); Julian V. Chan, Spring Valley, NY (US); Joseph M. Ting, Sudbury, MA (US)

Correspondence Address: Cummings & Lockwood, Granite Square, 700 State Street, P.O. Box 1960, New Haven, CT, 06509-1960, US

(73) Assignee: Medical Instill

(21) Appl. No.: 10/617,140

(22) Filed: Jul. 8, 2003

Related U.S. Application Data
(63) Continuation-in-part of application No. 10/294,926, filed on Nov. 14, 2002.
(60) Provisional application No. 60/394,618, filed on Jul. 8, 2002.
(60) Provisional application No. 60/396,514, filed on Jul. 16, 2002.

(51) Int. Cl. A61M 31/00

(52) U.S. Cl. 604/506; 604/117; 604/176

(57) ABSTRACT

A device for intradermal delivery having a housing containing a syringe for injecting a substance at a predetermined insertion depth and a base assembly for tensioning a target area of skin. One embodiment tensions the skin by vacuum and another by expansion of a mounting surface in contact with the skin. Upon tensioning the target area, a needle extends into the target area to the insertion depth. At the insertion depth, a plunger depresses within the syringe and delivers the substance therein through the needle. Upon completing injection, the needle retracts, the skin tensioning is released and the intradermal delivery device is removed.

### CROSS-REFERENCE TO RELATED APPLICATIONS

This patent application is a continuation-in-part of co-pending U.S. patent application Ser. No. 10/294,926, filed Nov. 14, 2002, entitled “Intradermal Delivery Device”, and claims priority on U.S. provisional patent application serial No. 60/394,618, filed Jul. 8, 2002, entitled “Intradermal Delivery Device, And Method Of Intradermal Delivery”, and U.S. provisional patent application serial No. 60/396,514, filed Jul. 16, 2002, entitled Intradermal Delivery Device Adhesively Attachable To The Skin, And Method Of Intradermal Delivery”, each of which is hereby expressly incorporated by reference as part of the present disclosure.

### BACKGROUND OF THE INVENTION

1. Field of the Invention

The subject invention relates to devices and methods for injecting a substance into a person or animal, and more particularly, to an improved device and method for injecting the substance intradermally.

2. Background of the Related Art

Drug delivery into the soft tissue inside the dermis, i.e., intradermal delivery, with a very small needle has been shown to slow drug release time and reduce or eliminate nerve ending stimulation and hence patient reaction. The challenge to delivering drugs in this fashion include the need for precise control over needle penetration depth which can vary due to tissue compliance and penetration angle.

Techniques have been developed to improve the ability of individuals to administer injections. For example, U.S. Pat. No. 4,393,870 to Wagner shows a suction injector for use by a patient. The suction injector of Wagner includes a medicine containing syringe sliceable attached to an outer chamber. The outer chamber is a sealed vacuum chamber. An inner chamber is concentric and sealingly isolated with respect to the outer chamber. The inner chamber receives the syringe. A membrane maintains the sterility of the syringe and a bellows placed circumferentially about the outer chamber prevents the syringe from piercing the membrane. In use, as the syringe slides in the outer chamber, the bellows retract and the vacuum seal between the inner chamber and outer chamber is broken. The skin is lifted under the resulting negative pressure and the medicine can be injected therein.

Some needle insertion devices, such as the device shown in U.S. Pat. No. 4,299,219 to Norris, Jr., have recognized that vacuum on the skin can increase the size of an underlying vein to facilitate locating the vein with the cannula. There are problems associated with the systems of Wagner and Norris, Jr., however. Both devices distort the surface of the skin in a calotte-shaped manner. The distortion creates a difficulty in controlling the insertion depth of the cannula. U.S. Pat. No. 6,200,291 to Di Pietro shows a needle surrounded by a distal end of a skin contacting element. The distal end is conic shaped and deforms when pressed against the skin. When deformed, the needle extends beyond the skin contacting element into the patient's skin. The limited ability of the distal end to deform limits the insertion depth of the needle. Although limited, the device of Di Pietro requires deft control by the operator to provide consistent insertion depth. Microholes in the conic distal end prevent a vacuum effect so the device can be easily removed after injection.

There is a need, therefore, for an improved intradermal delivery device and method that repeatably provide a definite relative skin state for precise needle penetration and reduced negative patient reaction.

### SUMMARY OF THE INVENTION

One aspect of the present invention is directed to an intradermal delivery device (“IDD”) and method for injecting a substance into the skin. The intradermal delivery device comprises a housing including a base defining a needle aperture, and a skin-engaging surface extending about a periphery of the needle aperture. A syringe of the intradermal delivery device includes a syringe body coupled to the housing and a plunger slidably received within the syringe body. A needle is coupled in fluid communication with the syringe body, and is movable through the needle aperture to penetrate the skin and inject a substance contained within the syringe body therein. A vacuum chamber of the intradermal delivery device is coupled in fluid communication with the base for drawing a vacuum within the base and, in turn, releasably securing the skin-engaging surface to the skin and forming a substantially planar needle penetration region on the skin. The intradermal delivery device further includes at least one stop surface fixed relative to at least a portion of the skin-engaging surface to define a predetermined distance therebetween, and adapted to cooperate with the needle to limit a depth of insertion of the needle into the needle penetration region of the skin. The needle is movable through the needle aperture upon slidably moving the plunger through the syringe body to thereby penetrate with the needle the penetration region of the skin and inject a substance contained within the syringe body therein.

In one embodiment of the present invention, the device further comprises a needle cap mounted over the needle and forming an approximately airtight seal therebetween, and defining a penetrable surface formed adjacent to the needle tip for passage of the needle therethrough. Preferably, the syringe, needle and needle cap form a sealed, pre-fillable subassembly insertable into the housing after filling the syringe body with a substance. Also in this embodiment of the present invention, the needle is a non-coring needle defining a closed end surface and at least one aperture located in a side wall thereof in fluid communication with the syringe body.

In another embodiment of the present invention, the device further comprises a pair of first and second finger grips formed on the housing on approximately opposite sides of the plunger relative to each other for receiving digits of a first hand. In addition, a third finger grip is formed on the housing adjacent to the base for receiving a digit of a second hand for controlling application of the intradermal delivery device to the skin.

In one embodiment of the present invention, the base defines at least one aperture formed adjacent to the skin-engaging surface and coupled in fluid communication with the vacuum chamber for drawing a vacuum through the aperture and releasably securing the skin-engaging surface to the skin. Preferably, the aperture extends adjacent to a periphery of the skin-engaging surface. In one embodiment of the present invention, the base defines at least one recess spaced on an opposite side of the vacuum aperture relative to the needle aperture and adapted to receive therein a sealant to facilitate the formation of a vacuum within the vacuum aperture and releasably secure the skin-engaging surface to the skin.

Another aspect of the present invention is directed to an intradermal delivery device, comprising a housing including a base defining a needle aperture and a skin-engaging surface extending about a periphery of the needle aperture. A syringe of the device includes a syringe body coupled to the housing and a plunger slidably received within the syringe body. A needle is coupled in fluid communication with the syringe body and is movable through the needle aperture to penetrate the skin and inject a substance contained within the syringe body therein. The device further includes at least one stop surface fixed relative to at least a portion of the skin-engaging surface to define a predetermined distance therebetween, and adapted to cooperate with the needle to limit a depth of insertion of the needle into the needle penetration region of the skin. The device also includes means for forming a substantially planar needle penetration region on the skin.

In one embodiment of the present invention, the means for forming a substantially planar needle penetration region on the skin is defined by at least a portion of the skin-engaging surface that is radially expandable. In this embodiment, the needle is movable through the needle aperture upon slidably moving the plunger through the syringe body to thereby penetrate with the needle the penetration region of the skin and inject a substance contained within the syringe body therein.

In another embodiment of the present invention, the means for forming a substantially planar needle penetration region on the skin is defined by a vacuum chamber coupled in fluid communication with the base for drawing a vacuum within the base and, in turn, releasably securing the skin-engaging surface to the skin and forming a substantially planar needle penetration region on the skin.

Another aspect of the present invention also is directed to a method for intradermal delivery, comprising the following steps:

providing an intradermal delivery device including a housing having a mounting surface and a reciprocally mounted syringe therein;

placing the mounting surface on the skin of a patient;

creating a vacuum between the housing and the skin and, in turn, releasably securing the mounting surface to the skin;

forming a substantially planar target penetration region on the skin;

introducing a needle of the syringe a predetermined depth into the substantially planar target penetration region of the skin; and

injecting a substance from the syringe through the needle into the substantially planar target penetration region of the skin.

In a currently preferred embodiment of the present invention, the method further comprises the steps of providing a non-coring needle defining at least one lateral opening in a side wall thereof; introducing the needle into the target penetration region of the skin at a predetermined depth wherein the at least one lateral opening is located substantially entirely within the derm; and injecting the substance laterally through the at least one opening of the needle and into the derma.

One advantage of the intradermal delivery device and method of the present invention is that the vacuum created by the device substantially prevents relative movement between the skin and the device, and thereby defines a substantially planar needle penetration region on the patient's skin facilitating insertion of the needle to a precise depth within the skin.

Other advantages of the intradermal delivery device and method of the present invention will become more readily apparent in view of the following detailed description of preferred embodiments and accompanying drawings.

### BRIEF DESCRIPTION OF THE DRAWINGS

So that those having ordinary skill in the art to which the disclosed device and method appertain will more readily understand how to make and use them, reference may be had to the drawings wherein:

FIG. 1A illustrates a perspective semi-transparent view of a device for injecting a substance intradermally in accordance with the present invention.

FIG. 1B illustrates a side semi-transparent view of the device of FIG. 1A.

FIG. 1C illustrates an end semi-transparent view of the device of FIG. 1A.

FIG. 1D illustrates multiple side and perspective line views and a cross-sectional view of the device of FIG. 1A.

FIG. 2A illustrates a front perspective, semi-transparent view of another embodiment of a device for injecting a substance intradermally in accordance with the present invention.

FIG. 2B illustrates a rear semi-transparent view of the device of FIG. 2A.

FIG. 3 is a cross-sectional view of the device of FIG. 2A.

FIG. 4 is another cross-sectional view of the device of FIG. 2A.

FIG. 5 is a perspective view of a tubular guide of the device of FIG. 2A.

FIG. 6 is a perspective view of a housing of the device of FIG. 2A.

FIGS. 7A, 7B and 7C are additional perspective views of the device of FIG. 2A.

FIGS. 8A, 8B and 8C are perspective views of three devices embodying the present invention that are configured to inject intradermally at 30°, 45°, and 60°, respectively.

FIGS. 9A, 9B, and 9C illustrate perspective views of another embodiment of a device embodying the present invention for intradermal delivery.

FIG. 9D illustrates a plurality of perspective views of the device of FIGS. 9A-C.

FIGS. 10A-10H are cross-sectional views of the device of FIG. 9A in various positions during injection of a medicament or other substance.

FIG. 11A is a cross-sectional view of the distal end of the device injecting a medicament or other substance into the skin of a patient.

FIG. 11B is an enlarged, localized cross-sectional view of the distal end of the needle of the device of FIG. 11A.

FIG. 12 is another cross-sectional view of the device of FIG. 9A.

FIG. 13 illustrates a plurality of perspective views of another embodiment of an intradermal delivery device of the present invention.

FIG. 14 is a cross-sectional view of the device of FIG. 13.

FIG. 15 is an enlarged localized view of one embodiment of a needle of the intradermal delivery device inserted in a patient's skin.

FIG. 16 is a cross-sectional view of another embodiment of an intradermal delivery device constructed in accordance with the present invention.

FIG. 17A is another cross-sectional view of the device of FIG. 16.

FIG. 17B is a side line view of the device of FIG. 16.

FIG. 18 is a perspective view of another embodiment of an intradermal delivery device of the present invention.

FIG. 19A is another perspective view of the device of FIG. 18.

FIG. 19B is another perspective view of the device of FIG. 18.

FIG. 19C is a localized perspective view of the device of FIG. 18.

FIG. 20 is a cross-sectional view of the device of FIG. 18.

FIG. 21A is an enlarged partial, cross-sectional view of the base of the device of FIG. 20 illustrating the grooves for receiving a lubricant, gel or like substance, that may or may not include an antiseptic and/or anti-bacterial substance, for facilitating the vacuum attachment of the device to a patient's skin and/or preventing infection.

FIG. 21B is an enlarged partial, cross-sectional view of the base of the device of FIG. 20 including an overmolded boot.

FIG. 22 is a perspective view of the housing of the device of FIGS. 18 and 19A.

FIG. 23A is a perspective view of the plunger of the device of FIGS. 18 and 19A.

FIG. 23B is a perspective view of the plunger of the device of FIG. 18.

FIG. 24 is an enlarged, partial cross-sectional view of a mounting surface, a needle mount, and a needle cap of the device of FIG. 18.

FIG. 25 is an enlarged, partial side elevational view of a non-coring needle tip of the device of FIG. 18.

FIG. 26 is an upper perspective view of the track follower of the device of FIG. 18.

FIG. 27 is a somewhat schematic, side elevational view of the housing of the device of FIG. 18 illustrating the pin and slot arrangement for controlling actuation of the device.

FIG. 28 is a top perspective view of the locking ring of the device of FIG. 18.

FIG. 29 is a perspective view of a syringe sub-assembly of the device of FIG. 18.

FIGS. 30-34 are sequential, perspective views illustrating operation of the device of FIG. 18.

FIG. 35 is a perspective view of another device that is configured for intradermal delivery and embodying the present invention.

FIG. 36 is a cross-sectional view of the device of FIG. 35 taken along line 36-36.

FIG. 37 is another cross-sectional view of the device of FIG. 35 taken along line 37-37.

FIG. 38 is an enlarged partial, cross-sectional view of the base of the device of FIG. 35 illustrating the tapered needle mount and expandable base for tensioning a patient's skin across the needle penetration region.

FIG. 39 is a perspective view of the housing of the device of FIG. 35.

FIG. 40 is a perspective view of another housing of a device that is configured for intradermal delivery and embodying the present invention.

### DETAILED DESCRIPTION OF THE PREFERRED EMBODIMENTS

The present invention overcomes many of the prior art problems associated with devices for intradermally injecting substances, such as vaccines, pharmaceutical, and cosmetic substances. The advantages, and other features of the devices and methods disclosed herein, will become more readily apparent to those having ordinary skill in the art from the following detailed description of certain preferred embodiments taken in conjunction with the drawings which set forth representative embodiments of the present invention and wherein like reference numerals identify similar structural elements.

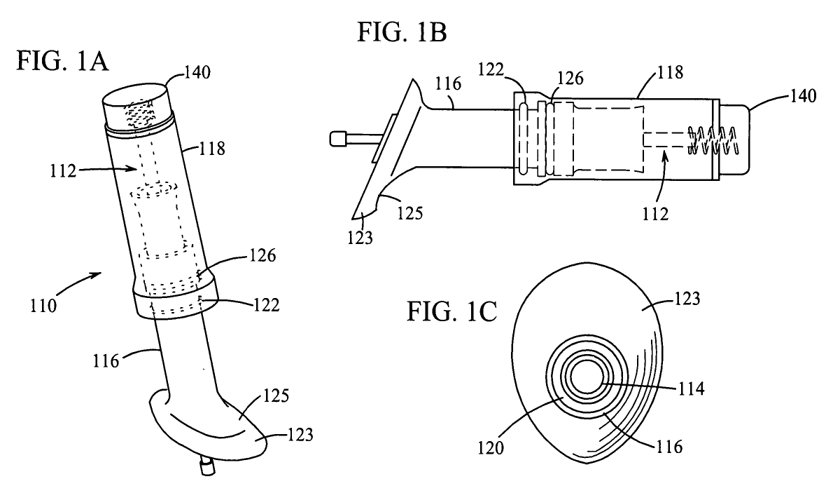

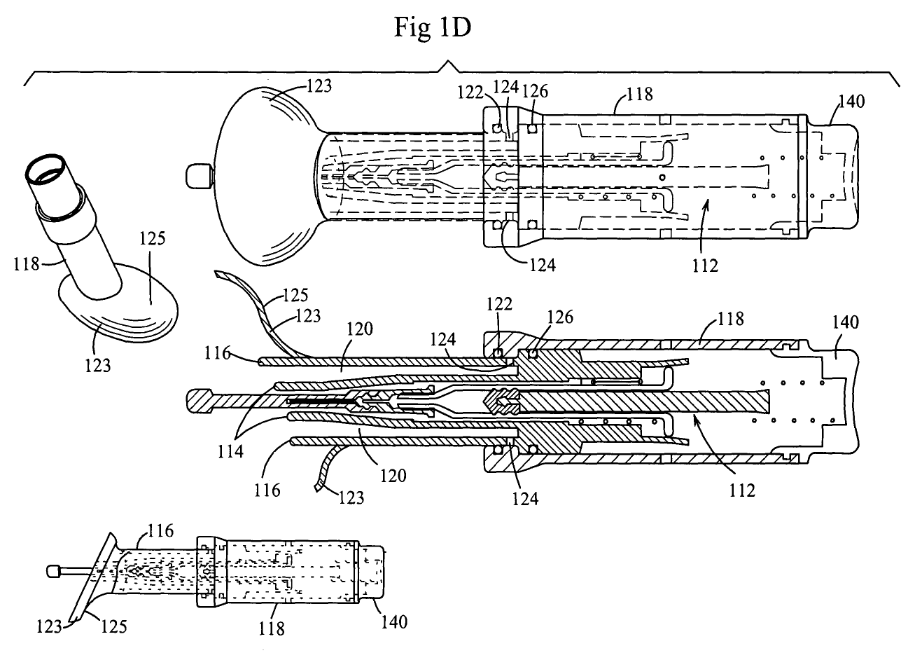

Referring now to FIGS. 1A, 1B, 1C and 1D, the subject device, referred to generally by reference numeral 110, provides for automatic needle orientation, penetration to a fixed depth for injection, and withdrawal in a single motion. After use, the device can be reloaded for subsequent use, if desired. As may be recognized by those of ordinary skill in the pertinent art based on the teachings herein, although the device 110 and other devices are described herein as intradermal delivery devices or “IDDs”, these and other devices embodying the present invention need not be limited to, or used solely for intradermal delivery, but rather such devices equally may be configured or otherwise employed to deliver medicaments or other substances in any of numerous other ways, such as by sub-cutaneous delivery.

The device 110 comprises a syringe 112 nested inside a first housing defining two concentric shells, an inner shell 114 and an outer shell 116. An elongated annular channel 120 is formed between the inner shell 114 and the outer shell 116. A relatively large base 123 surrounds the outer shell 116 for providing greater stability against the patient's skin. A depression area 125 in the base 123 accommodates the user's thumb for further stabilization. In operation, the inner shell 114 and the outer shell 116 are placed against the skin of the patient. While one hand holds the device 110, the thumb of the other hand can stabilize the skin interface by placement upon the depression area 125. As described further below, the distal end of the inner shell 114 defines a needle aperture allowing the needle end of the syringe 112 to pass therethrough. In addition, the distal end of the inner shell 114 defines a first skin-engaging surface extending about the periphery of the needle aperture, and the distal end of the outer shell 116 defines a second skin-engaging surface spaced radially outwardly relative to the first skin-engaging surface.

A second housing 118 receives the concentric shells 114, 116 in a sliding engagement. A first seal 122 on the distal end of the housing 118 forms a variable length channel defining a vacuum chamber that is coupled in fluid communication with the elongated annular channel 120 via ports 124 formed in the outer shell 116. It is envisioned that either a single port 124 or a plurality of ports may be used. A second seal 126 provides for airtight engagement of the proximal ends of the concentric shells 114, 116 and the housing 118. A threadably engaged cap 140 allows access within the housing 118 to install or replace the syringe 112 after use. As described further below, movement of the second housing 118 relative to the first housing defined by the concentric shells 114, 116, creates a vacuum within the variable-length channel and channel 120 to releasably secure the skin-engaging surfaces defined by the distal ends of the inner and outer shells 114, 116 to the skin, and form a substantially planar needle penetration region “X” on the skin. As shown typically in FIG. 1D, the distal end, or skin-engaging surface of the inner shell 114 is axially offset inwardly relative to the distal end, or skin-engaging surface of the outer shell 116 by a distance “A”, in order to allow the skin to move radially outwardly relative to the distal end of the inner shell 114 in response to the substantially radially directed forces exerted on the skin by the vacuum within the channel 120 to, in turn, facilitate formation of the substantially planar target penetration region “X” on the skin. In the illustrated embodiment of the present invention, the contact offset is determined by the distance between the substantially parallel planes defined by the distal ends, or skin-engaging surfaces of the inner and outer shells 114, 116. Further, as also described further below, the plane of each distal end or skin-engaging surface of the inner and outer shells 114, 116 is oriented at an acute injection angle “B” relative to a normal to the axis of the device. In the illustrated embodiment of the present invention, the contact offset is about 1.15 mm, and the injection angle B is about 25°; however, as may be recognized by those of ordinary skill in the pertinent art based on the teachings herein, the particular contact offset and injection angle of the illustrated embodiment are only exemplary, and numerous other offsets (or lack thereof) and/or injection angles equally may be employed.

As also described further below, the intradermal delivery device 110 further includes a stop surface 146 fixed relative to the skin-engaging surfaces 114, 116 to define a predetermined distance therebetween, and adapted to cooperate with a needle 137 of the syringe 212 to limit a depth of insertion of the needle into the needle penetration region X of the skin. The needle 137 is movable through the needle aperture defined by the skin-engaging surface or distal end of the inner shell 114 upon slidably moving a plunger 132 of the syringe through the syringe body to thereby penetrate with the needle the penetration region X of the skin and inject a substance contained within the syringe body therein. In one embodiment of the present invention, the IDD may enable the needle tip to be precisely located within a penetration zone of less than about 5 mm in depth, and preferably within the range of about 1 mm to about 3 mm in depth. In addition, the IDD of the present invention preferably enables such precise locating of the needle tip from one IDD to the next.

In the operation of the device 110, the inner shell 114 and the outer shell 116 are placed against the skin of the patient. As the cap 140 is depressed, the variable length channel expands to create a vacuum. The vacuum extends into the annular channel 120 through ports 124, and pulls the surface of the skin toward or into the channel 120. As a result, the skin is stretched tightly over the inner shell 114. The folds and contours of the skin along with tissue compliance, which can make penetration to a fixed depth difficult, are effectively and painlessly removed. Such tensioning results in the target area of the skin surface being a substantially flat, taut reference plane X. The skin tensioning also helps to ensure that the cannula 137 will always penetrate at a predetermined fixed angle with respect to the reference plane of the skin.

As the cap 140 is further depressed, the housing 118 slides down the outer shell 116 and a spring 142 fixedly secured to the underside of the cap 142 engages a flange 144 on the syringe body 128 at approximately the same time that the underside of the cap 140 contacts the proximal end of the plunger 132. A spring 138 biases the syringe body 128 inwardly, and is softer, or exerts a lesser spring force, than the spring 142 attached to the cap. Thus, the spring 138 compresses before the spring 142 begins to compress. As the cap 140 is depressed further, the softer spring 138 significantly deforms until the flange 144 makes contact with the hard stop 146. The hard stop 146 limits the distance that the syringe 112 extends. As a result, the cannula 137 of the syringe 112 penetrates the skin to the same predetermined depth during each use. The medicament is not expelled before complete insertion of the cannula 137 to the predetermined depth.

Then, as the cap 140 is further depressed, the spring 142 begins to deform and the plunger 132 is inserted into the cavity 130 of the syringe 112. As a result, the plunger 132 expels the dosage out of the needle 137 and into the skin of the patient. The insertion of the plunger 132 into the syringe body 128 is limited by a sleeve 141 of the cap 140 contacting a shoulder 115 formed by the inner shell 114. Preferably, the sleeve 141 and shoulder 115 are sized and configured to determine the dosage of substance released from the syringe 112. At the end of the stroke of the cap 140, a plurality of relief holes 150 formed in the housing 118 vent the variable length channel to ambient and, in turn, release the vacuum on the skin to allow removal of the device 110. Thus, the device 110 operates in one single motion which can be performed without the aid of a second person. In order to prepare for another injection, the cap 140 is removed from the housing 118. The used needle syringe 112 is removed and replaced with a new full needle syringe 112.

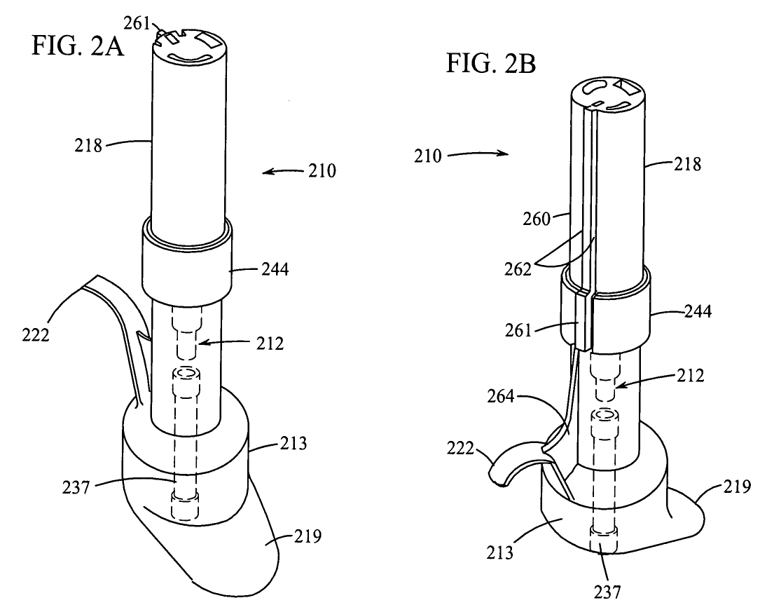

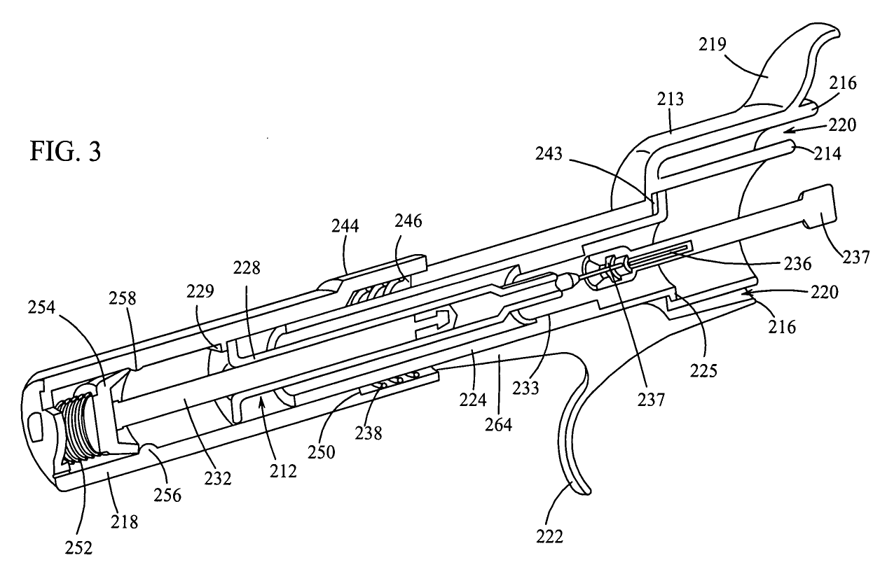

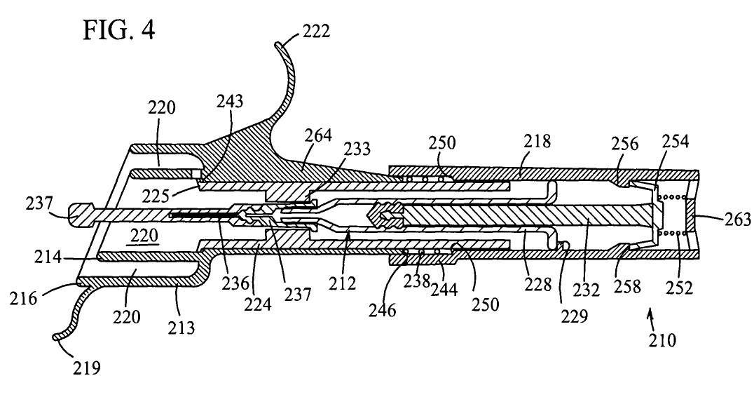

Referring to FIGS. 2A, 2B, 3 and 4, the subject device, referred to generally by reference numeral 210, also provides for automatic needle orientation, penetration to a fixed depth, and withdrawal in a single motion. After use, the device can be reloaded for subsequent use. For simplicity of description, an effort has been made to denote similar parts between device 110 and device 210 with reference numerals having a “2” for the first digit instead of a “1”. The device 210 comprises a syringe assembly 212 concentrically located within a the base of a first housing or barrel assembly 213. As shown best in FIGS. 3 and 4, the barrel assembly 213 includes an inner barrel 214 and an outer barrel 216. An elongated annular channel 220 is formed between the inner barrel 214 and the outer barrel 216. In another embodiment (not shown), the inner barrel 214 and outer barrel 216 terminate in a soft tip or gasket for improved comfort and sealing performance. An integral thumb rest 219 facilitates stabilization of the angled distal portion of the barrel assembly 213 against the patient's skin. A trigger grip 222 provides a location for a finger of the user to grip for additional control of the device 210.

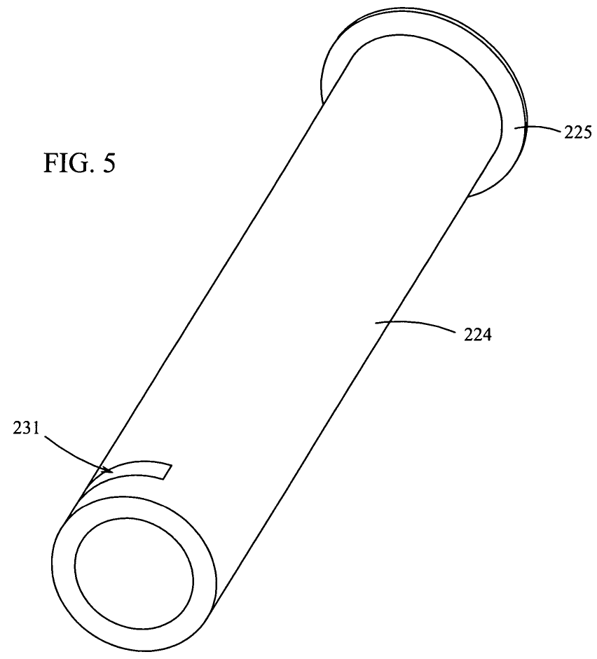

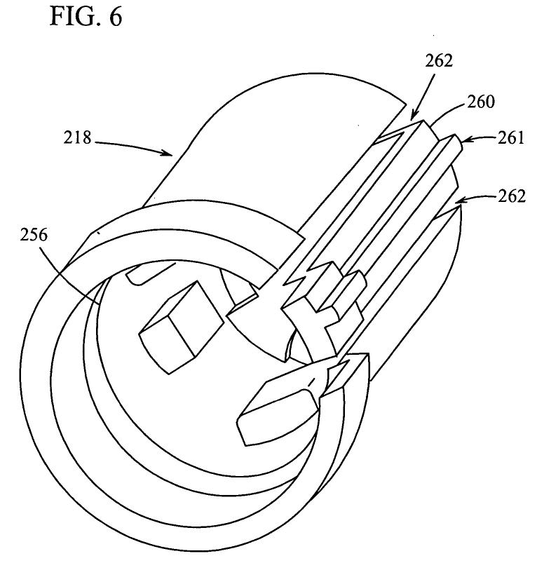

Referring now to FIGS. 3 and 4, a second or tubular housing 218 receives the barrel assembly 213 in a sliding engagement. The syringe assembly 212 is held partially within the housing 218 and partially within the barrel assembly 213. The syringe assembly 212 has a tubular guide 224 which is coupled to the housing 218 for sliding therewith. As best seen in FIG. 5, the tubular guide has a slot 231 for coupling to a protrusion (not shown) of the housing 218. The distal end of the tubular guide 224 has a contact seal 225 for creating a variable space 223 in communication with the annular channel 220. A port or a plurality of ports 227 in the inner barrel 214 allow air to pass between the variable space 223 and channel 220. As can be seen, the contact seal 225 is dimensioned to slidably contact the interior surface of the inner barrel 214 and form a gas-tight seal therebetween. In the illustrated embodiment of the present invention, the contact seal and the inner barrel are each formed of a thermoplastic material selected to create a gas-tight, plastic-on-plastic seal between the contact seal and inner barrel, and thereby obviate the need for an additional o-ring or other sealing member, as described, for example, in connection with the device 110 above. If desired, an o-ring or other seal (not shown) may be located between the barrel assembly 213 and tubular guide 224, and above the inner barrel 214 and the outer barrel 216 for sealing the proximal end of the variable space 223.

Referring now to FIG. 4, preferably, the syringe assembly 212 is of a conventional design. A plunger 232 on the syringe assembly 212 slidably penetrates a body 228 for forcing a medicament out of a needle 236. A needle-mounting member 237 secures the needle 236 to the body 228. The syringe assembly 212 is retained between a protrusion 229 on the housing 218 and a shoulder 233 on the tubular guide 224. As a result, the housing 218, the syringe 226 and tubular guide 224 are all linked together and the relationship is maintained during compression of the device 210. As shown, when filled with a medicament or other substance and in a storage position, a removable cap 239 covers the needle 236.

For storage, a spring 238 biases the housing 218 away from the barrel assembly 213, i.e., in a retracted needle position. An enlarged diameter distal portion 244 of the housing 218 retains the spring 238. To extend the needle 236, the spring 238 is compressed between the proximal end 246 of the barrel assembly 213 and a transitional shoulder portion 250 of the housing 218.

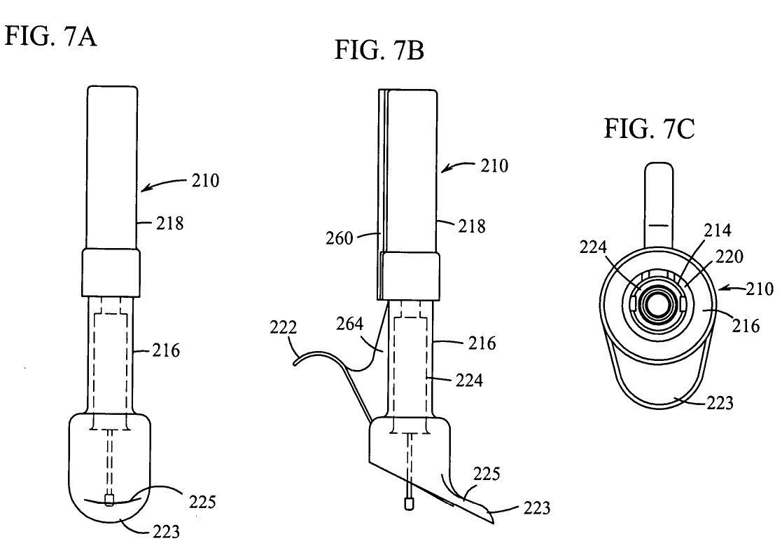

A second spring 252 provides a force to depress the plunger 232. In the storage position, the spring 252 is compressed within the proximal portion of the housing 218 by a spring stop 254. The spring stop 254 rests on shoulders 256, 258 integral with housing 218. As shown in FIGS. 4 and 6, one shoulder 258 is located on a camming portion 260 of the housing 218. As shown in FIG. 6, slots 262 and a flex point 263 in the proximal end of the housing 218 allow the camming portion 260 to expand in diameter and, when expanded, the spring stop 254 can pass by the shoulders 256, 258. A ridge 261 (see FIGS. 2A and 6) provides stiffening so as to increase the pressure on, and thereby the flexing of the flex point 263. An upstanding flange 264 on the barrel assembly 213 forces the expansion of the camming portion 260 as the housing 218 is depressed over the barrel assembly 213. FIGS. 7A, 7B and 7C show additional views of device 210.

With reference to FIG. 4, in operation, the proximal end of the device 210 is placed in the palm of the hand of the user. A digit on the same hand, preferably the forefinger or middle finger, grips the trigger grip 222 to provide control of the device. The trigger grip 222 further provides a leverage point to allow easy compression of the device 210 without exerting undue force against the skin of the patient. The distal ends or skin-engaging surfaces of the inner barrel 214 and the outer barrel 216 are placed against the skin of the patient to effectively seal the channel 220. While one hand holds the device 210, the thumb of the other hand can further stabilize the skin interface by placement within the thumb rest 219. Compression of the housing 218 upon the barrel assembly 213 forces the contact seal 225 along the inner barrel 214, thereby expanding the size of the variable space 223 therebetween. Due to the effective sealing of the channel 220, the expanding variable space 243 creates a vacuum which generates a vacuum in the channel 220 as well. As a result, the skin is tensioned within the channel 220 by vacuum and, thereby, tensioned across the inner barrel 214 to create a substantially planar reference plane for the needle 236 to penetrate. As shown in FIG. 4, the distal end, or skin-engaging surface of the inner shell 214 is axially offset inwardly relative to the distal end, or skin-engaging surface of the outer shell 216 by a distance “A” in order to allow the skin to move radially outwardly relative to the distal end of the inner shell 214 in response to the substantially radially directed forces exerted on the skin by the vacuum within the channel 220 to, in turn, facilitate formation of the substantially planar target penetration region “X” on the skin.

The insertion depth of the needle 236, i.e. the distance the needle 236 extends beyond the inner barrel 214 into the tensioned skin, is determined by the proximal end 246 of the barrel assembly 213 in cooperation with the shoulder 250 of the housing 218 and spring 238. More specifically, axial movement of the housing 218 toward the barrel assembly 213 causes the shoulder 250 of the housing to compress the spring 238 against the proximal end 246 of the barrel assembly. Simultaneously, the protrusion 229 of the housing 218 drives the syringe body 228 axially outwardly of the device and, in turn, drives the needle 236 of the syringe toward the needle aperture defined by the skin-engaging surface or distal end of the inner barrel 214. As shown in FIG. 4, the needle mounting member 237 of the syringe 212 defines a peripheral flange 241 that axially engages the shoulder 233 on the tubular guide 224 to cause the guide to move axially with the syringe. Accordingly, as the housing 218 is moved inwardly toward the barrel assembly 213, the syringe axially drives the tubular guide 224 and contact seal 224 thereof outwardly to, in turn, increase volume of the variable volume chamber 223, create a vacuum in the channel 220, and releasably attach by vacuum the skin-engaging surfaces of the inner and outer barrels 214, 216 to the patient's skin and form the substantially planar target penetration region thereon. As the spring 238 becomes fully compressed between the proximal end 246 of the barrel assembly and shoulder 250 of the housing, the insertion depth of the needle 236 is achieved, and the camming portion 260 of the housing 218 is flexed outwardly such that the spring stop 254 is released from the shoulders 256, 258 of the housing. Thus, the proximal end 246 of the barrel assembly defines a stop surface fixed relative to the skin-engaging surfaces of the barrel assembly to define a predetermined distance therebetween, and adapted to cooperate with the needle 236 to limit a depth of insertion of the needle into the needle penetration region X of the skin. In another embodiment, the barrel assembly 213 includes a protrusion (not shown) on the upstanding flange 264 which further extends the camming portion 260 coincident with the full compression of the spring 238 to facilitate release of the spring stop 254 when the needle 236 is at the insertion depth. The release of the spring stop 254 allows the second spring 252 to axially drive the plunger 232 of the syringe inwardly until the plunger tip engages the base of the syringe body 228 to thereby inject the medicament or other substance contained within the chamber of the syringe body through the needle tip and into the skin. One advantage of the illustrated embodiment of the present invention is that the second spring 252 delivers a substantially constant force for axially moving the plunger 232 and injecting the medicament or other substance into the skin. Thus, the medicament or other substance may be delivered into the subject at a substantially constant, patient-independent rate.

Upon injection of the medicament, the user releases the compressive force upon the device 210, and the spring 238 forces the housing 218 back to the storage position thereby extracting the needle 236 from penetration. As the housing retracts, the contact seal 225 returns along the inner barrel 214, thereby decreasing the size of the variable space 223 therebetween. As the variable space 223 is minimized, the vacuum created therein is removed. As a result, the skin is released from the channel 220 and the device 210 is easily removed.

In one embodiment of the present invention, the needle 236 is beveled at an angle to maximize the area of the exit aperture thereof within the derma. Further, the arrangement of the currently preferred embodiments orients the needle 236 to correspond most effectively with the angle at which the skin of the patient is tensioned or rendered taut. In one embodiment, the arrangement for orienting the needle 236 is a series of mechanical keys (not shown). For example, a key on the needle-member 237 may indicate an orientation of the bevel angle. Such member key is received in a cavity (not shown) on the syringe body 228 which, in turn, has another key-cavity pair to reference the body 228 to the tubular guide 224 which, in turn, has another key-cavity pair to reference the tubular guide 224 to the barrel assembly 213. Consequently, the orientation of the bevel of the needle 236 is set with respect to the angle of the barrel assembly 213.

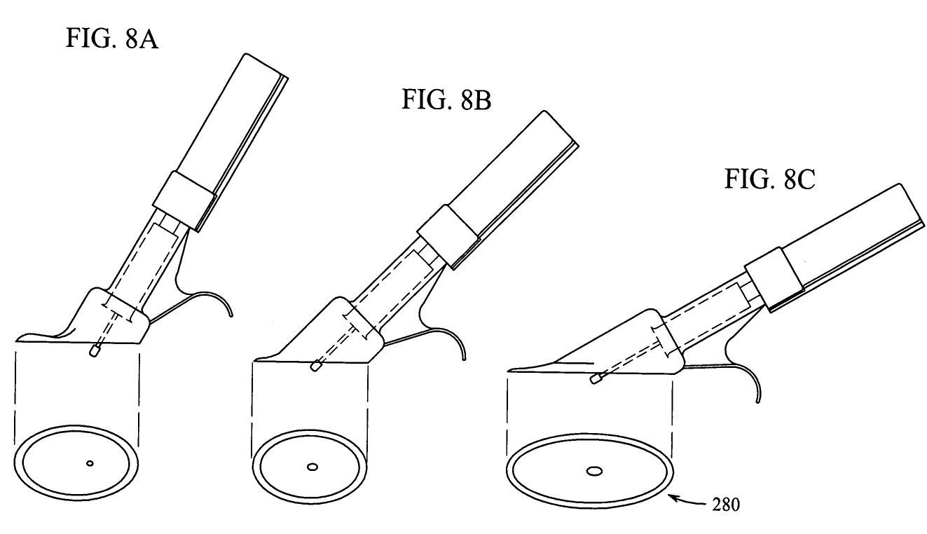

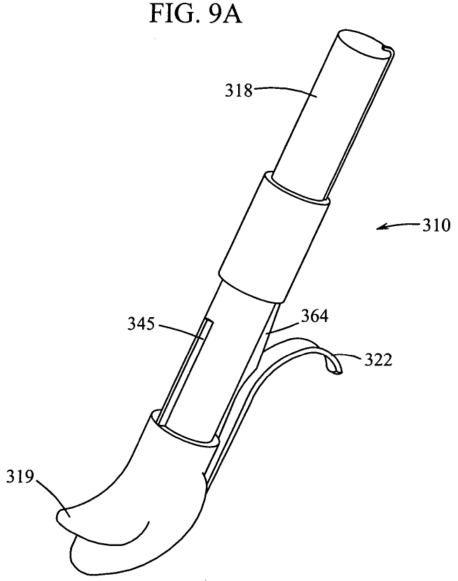

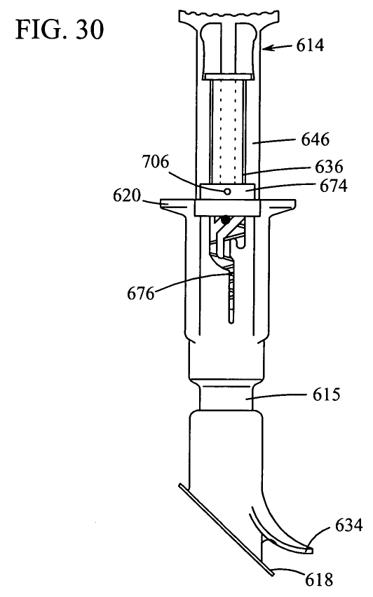

Referring to FIGS. 8A, 8B and 8C, 30 degree, 45 degree and 60 degree variations of the angle of the barrel assembly are shown, respectively. As the angle is increased, the surface area of the tensioned skin increases. As a result of the increased surface area, a larger amount of vacuum may be required and the parameters of the shown embodiments may be adjusted to optimize performance as would be appreciated by those of ordinary skill in the pertinent art based upon review of the subject disclosure. Additionally, as best seen in FIG. 8C, as the angle increases the portion of the elongated channel 220 which acquires vacuum on the patient's skin becomes an elongated oval 280, even though the barrel assembly is circular. To the extent that an oval vacuum area may yield uneven tensioning of the skin, the shape of the barrel assembly can be changed to an elongated shape normal to the otherwise oval vacuum area to yield an approximately circular shape to the vacuum area, if desired.

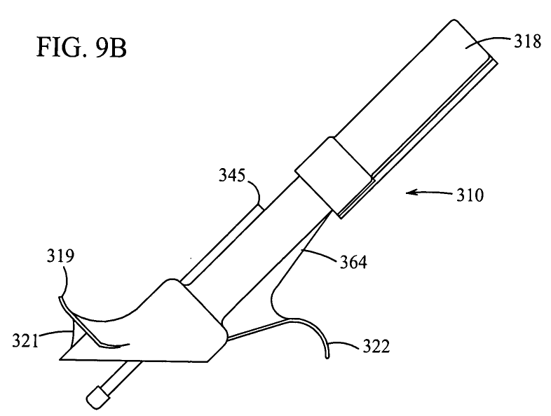

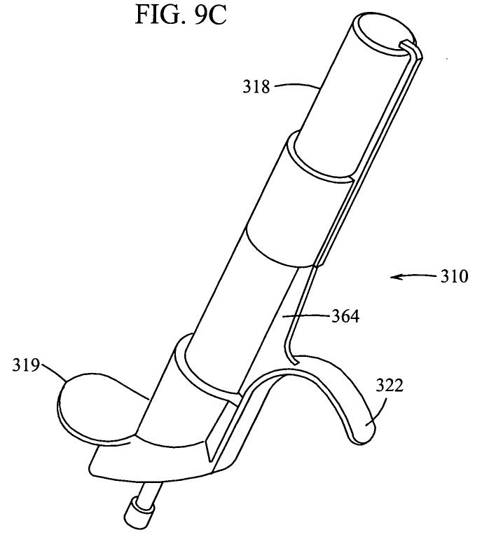

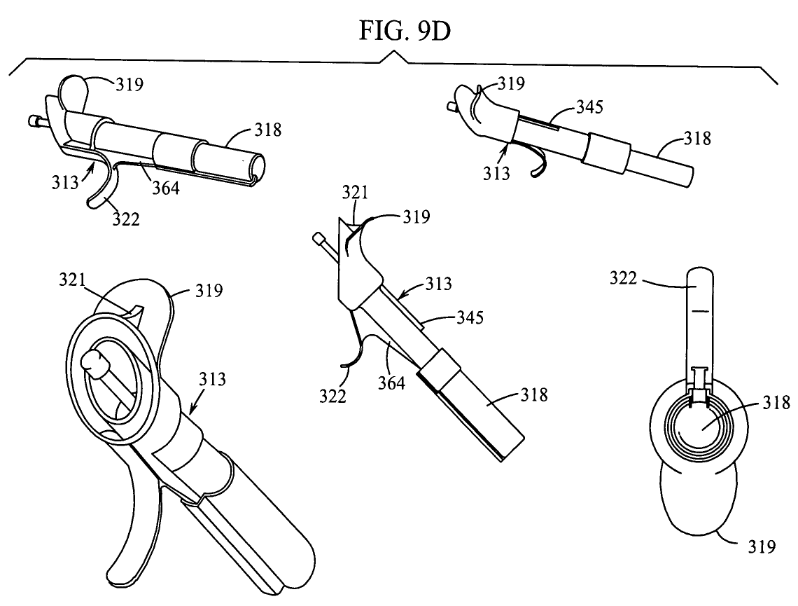

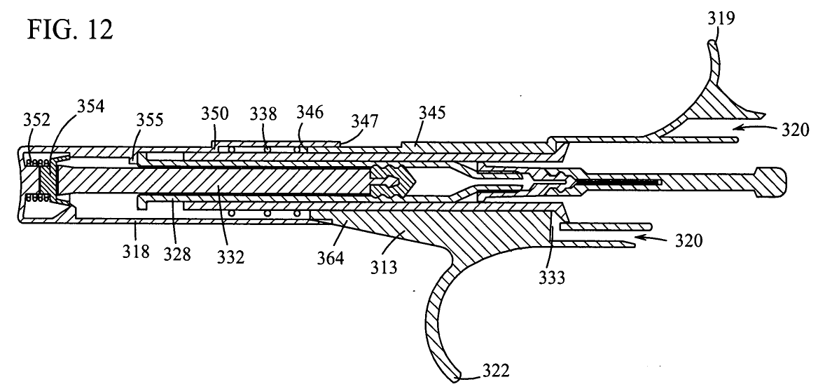

As shown in FIGS. 9A-12, another embodiment of the intradermal delivery device, referred to generally by the reference numeral 310, is shown. For simplicity of description, an effort has been made to denote similar parts between device 310 and device 210 with reference numerals having a “3” for the first digit instead of a “2”. Moreover, the following detailed description is largely related to the differences between device 310 and device 210; however, it will be appreciated by those of ordinary skill in the pertinent art that the inventive concept illustrated and described is clearly enabled, and practicing the advantages of the same is well within the skill of those of ordinary skill in the pertinent art upon review of the subject disclosure.

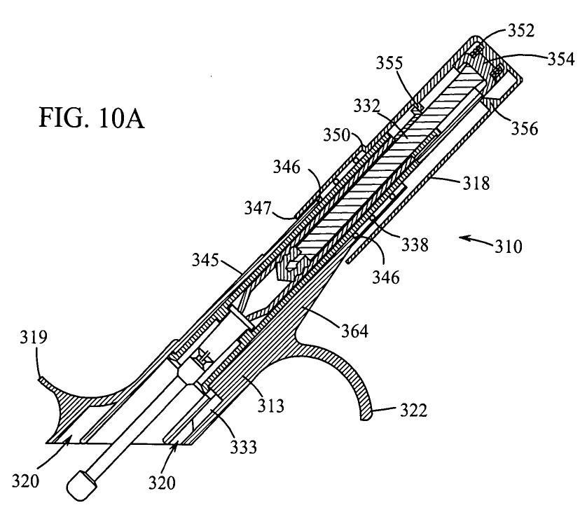

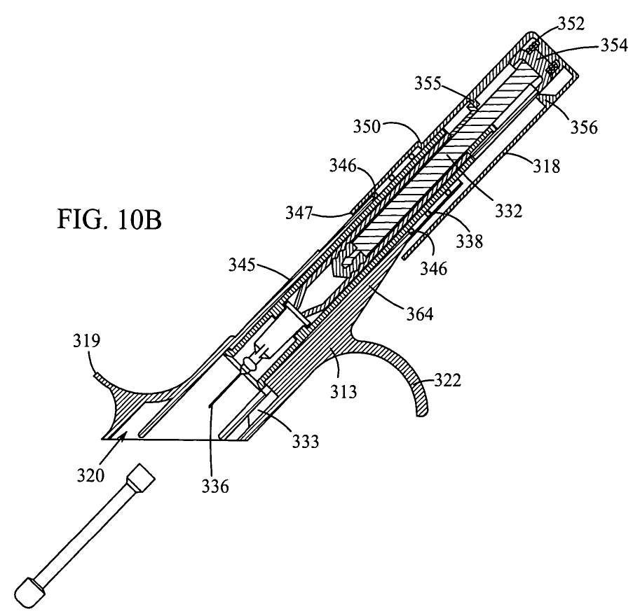

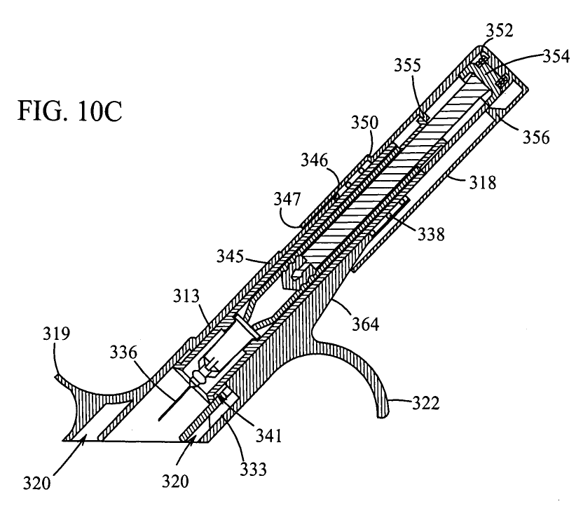

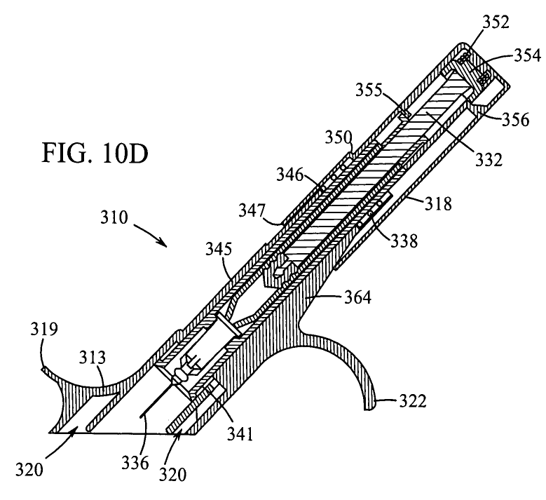

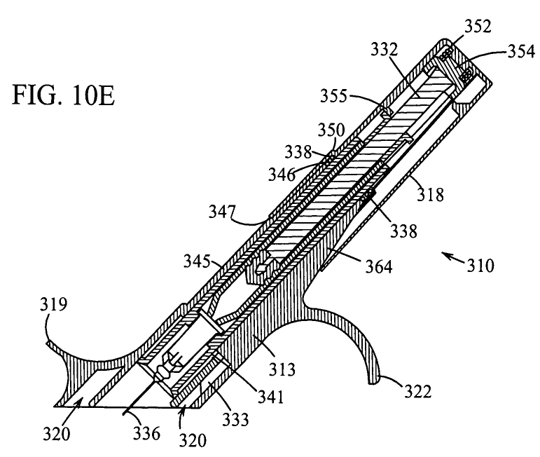

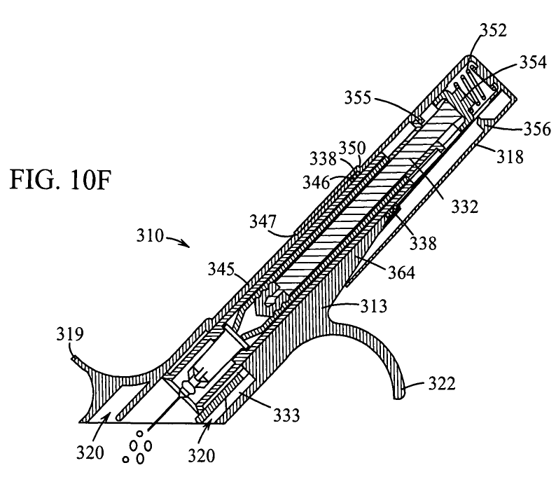

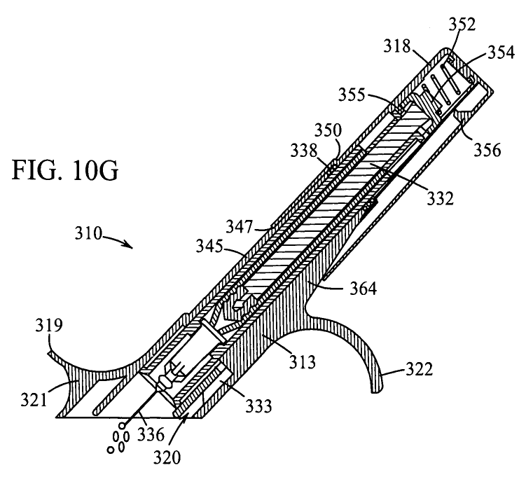

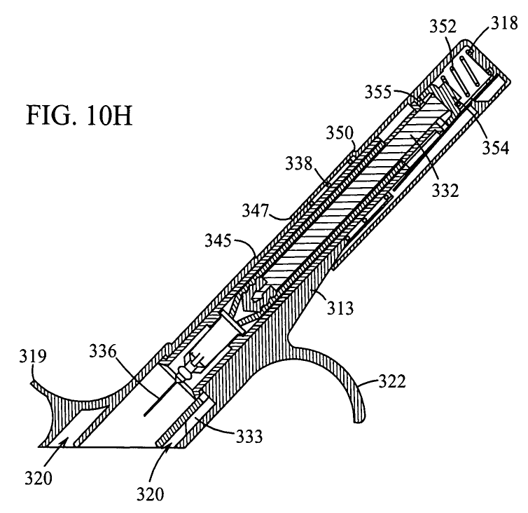

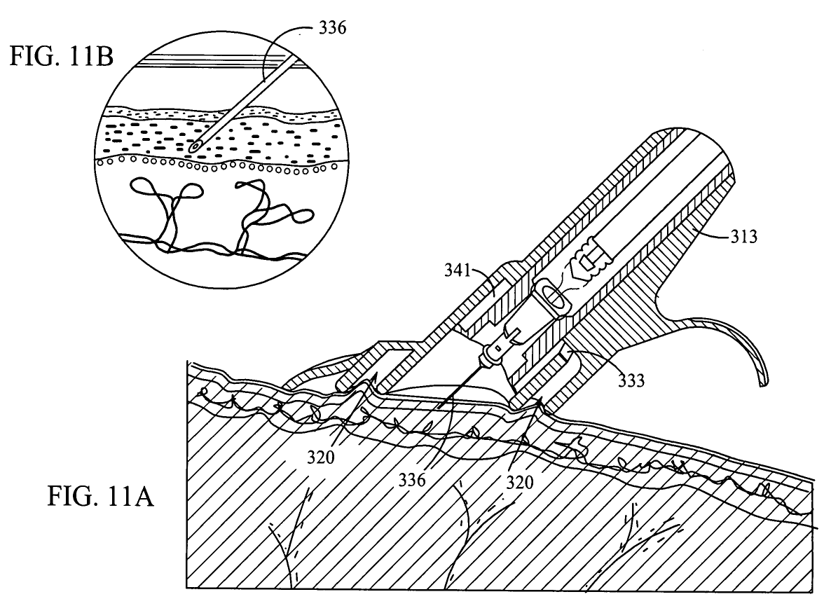

Referring now to FIGS. 9A-9D, the thumb rest 319 for stabilizing the device 310 against the skin of the patient includes a support rib 321 to stiffen the thumb rest 319. As best shown in FIGS. 10A-10H, the stroke limiting arrangement includes a barrel assembly 313 having an upstanding ridge 345 for engaging a distal end 347 of the housing 318. The interface between the upstanding ridge 345 and distal end 347 is preferably defined by two hard surfaces to create a repeatable and predictable extension of the needle 336 (see FIG. 10B) into the skin. A spring 338 extends between a shoulder 350 of the housing 318 and the proximal end 346 of the barrel assembly 313 to bias the housing 318 toward a storage position, as shown in FIG. 10A.

Still referring to FIGS. 10A-10H, compared to device 210, the vacuum area 320 of the barrel assembly 313 is reduced in order to increase the amount of vacuum created therein. A passageway 333 connects the variable space 341 (see FIG. 10C) to the vacuum area 320 for communicating the vacuum therebetween. As best seen in FIG. 10G, the travel of the spring stop 354 is limited by shoulder 355. In an alternative embodiment, the travel of the spring stop 354 is limited by the depth to which the plunger 332 can extend into the body 328. FIGS. 10G, 11A and 11B illustrate additional views of the device 310 while injecting a substance intradermally. Otherwise, the operation of the device 310 is the same, or substantially the same, as the operation of the device 210 described above.

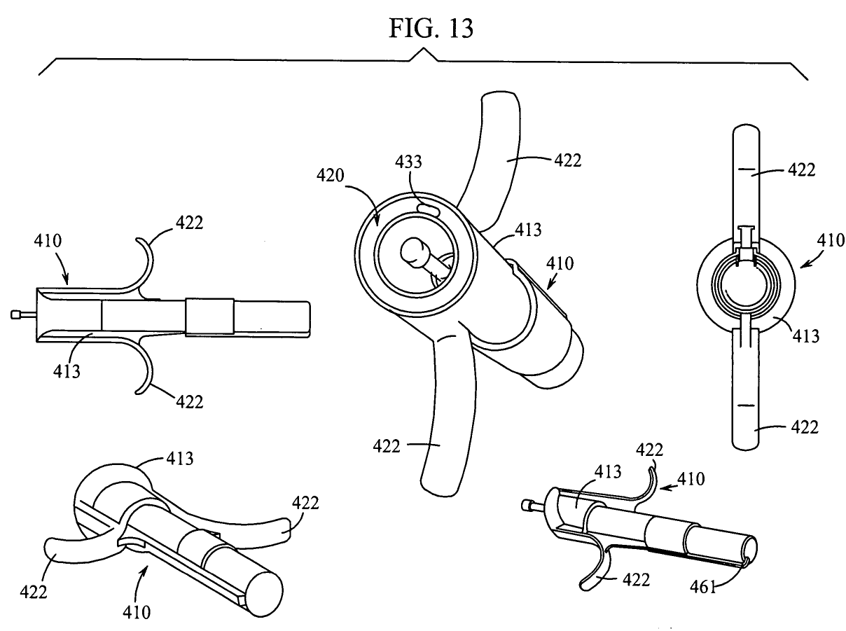

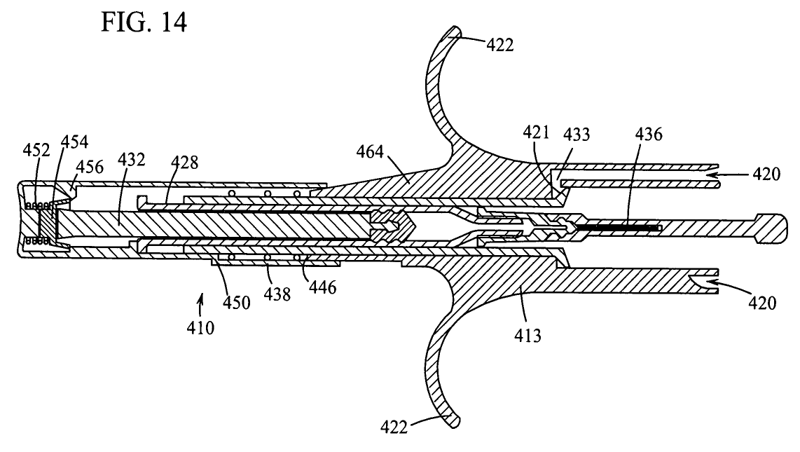

Referring to FIGS. 13 and 14, another embodiment of an intradermal delivery device, referred to generally by reference numeral 410, is illustrated. As will be appreciated by those of ordinary skill in the pertinent art, the device 410 utilizes many of the same principles of the devices 110, 210 and 310 described above. Accordingly, like reference numerals preceded by the numeral “4” instead of the numerals “1”, “2” or “3”, respectively, are used to indicate like elements whenever appropriate. In addition, whenever appropriate the description herein is largely directed to the differences for simplicity.

The barrel portion 413 of device 410 is designed for penetration of the needle 436 at an angle generally perpendicular to that of the skin of the patient. Although it would still be advantageous, the device 410 does not have a thumb rest; instead, the device 410 has two trigger grips 422. The vacuum channel 420 extends annularly between the inner skin-engaging surface 414 and the outer skin-engaging surface 416, and is coupled through an opening 433 with the axially-extending portion of the channel 420 in communication with the variable space 421.

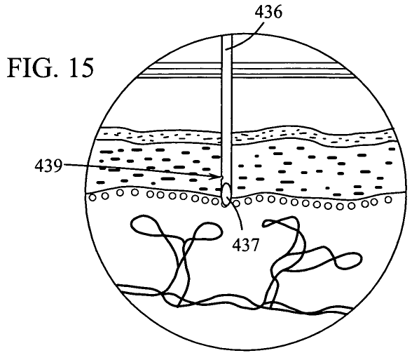

Turning to FIG. 15, preferably a needle 436 with an occluded tip 437 is deployed in device 410. The non-coring needle 436 has an angled bezel to effectively and relatively painlessly penetrate the skin. An outlet 439 allows release of the medicament from the passageway within the needle 436. It is envisioned that a plurality of apertures may be provided in the needle 436 to effectuate quicker release of the medicament or other substance. Preferably, for intradermal deliveries of medicament, the tip 437 of the needle 436 is below the dermis so that the aperture 439 is positioned optimally therein.

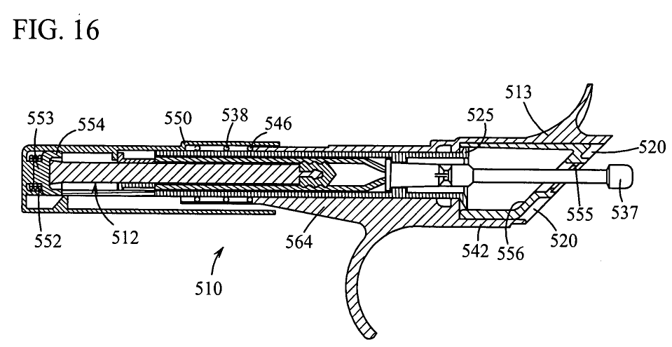

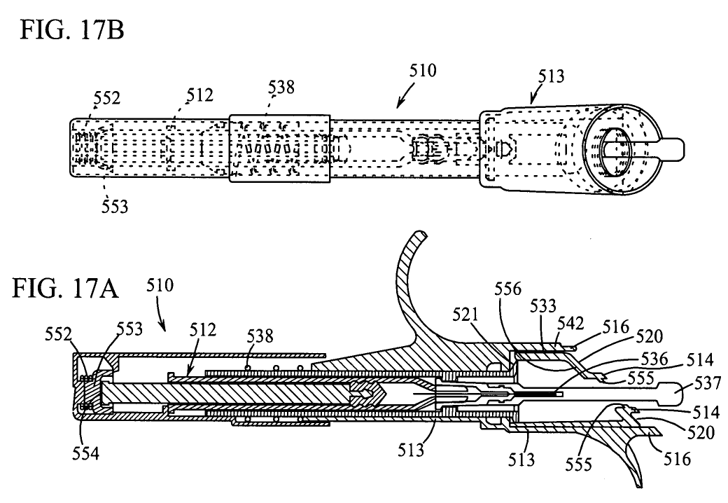

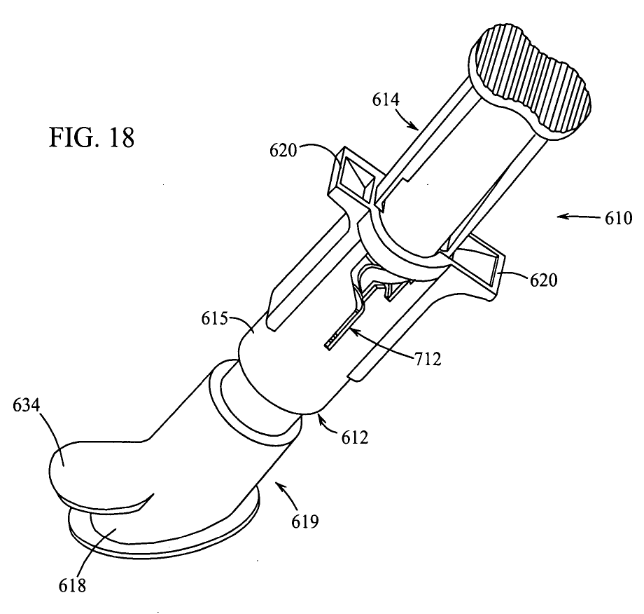

Referring now to FIGS. 16, 17A and 17B, another embodiment of an intradermal delivery device, referred to generally by reference numeral 510, is illustrated. As will be appreciated by those of ordinary skill in the pertinent art, the device 510 utilizes many of the same principles of the device 410 described above. Accordingly, like reference numerals preceded by the numeral “5” instead of the numeral “4” are used to indicate like elements whenever appropriate. In addition, whenever appropriate the description herein is largely directed to the differences for simplicity.

The shell assembly 513 has a ridge 542 for stiffening the outer shell 516 when applied to the skin. The outer shell 516 and inner shell 514 define vacuum area 520 of the shell assembly 513. The vacuum area 520 can be modified to tension more or less skin by changing the shape of the inner shell 514. An annular grid 555 on the inner side of the inner shell 514 prevents bulging of the skin in the area of penetration of the needle 536. A port 533 is formed between the annular-extending portion and axially-extending portions of the vacuum chamber 520.

Alternatively, if desired, the stroke limiting arrangement may utilize an angled surface 556 of the inner shell 514. As the contact seal 525 moves toward the skin, the seal 525 is limited by angled surface 556 to create a repeatable and predictable extension of the needle 536 into the skin. The spring stop 554 includes a top hat portion 553 for maintaining the orientation of spring 552.

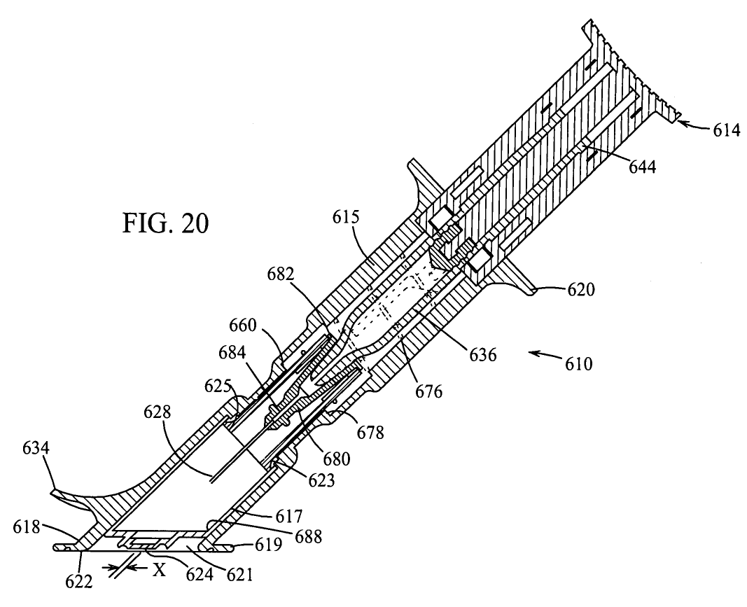

In FIGS. 18-20, another device embodying the present invention is indicated generally by the reference numeral 610. As will be appreciated by those of ordinary skill in the pertinent art, the device 610 utilizes many of the same principles of the devices described above. Accordingly, like reference numerals preceded by the numerals “6” or “7” instead of the preceding numerals are used to indicate like elements whenever appropriate. In addition, whenever appropriate the description herein is largely directed to the differences for simplicity. The device 610 comprises a housing body 615 and a syringe 614 mounted within the housing body 615. The housing body 615 defines a hollow interior 616 (FIG. 20), a base 618 formed at one end of the housing, and a pair of diametrically-opposed, first finger grips 620 formed at the other end of the housing.

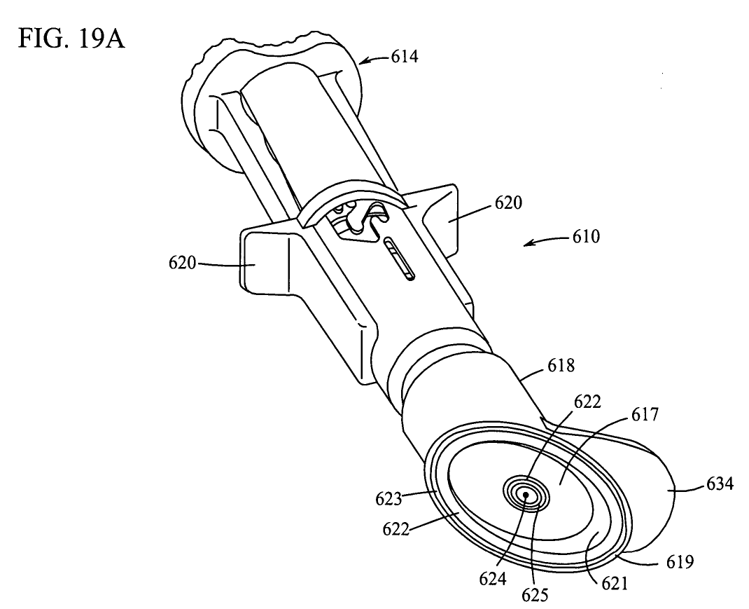

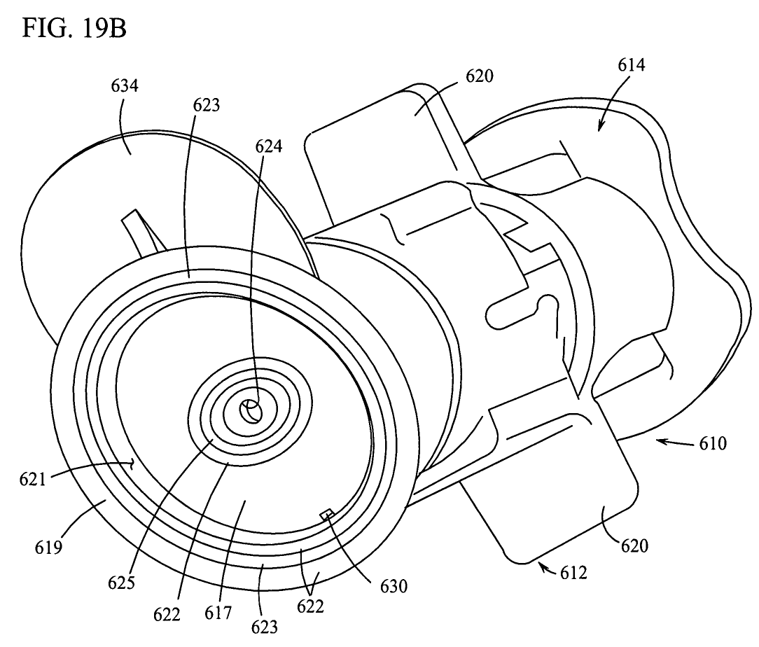

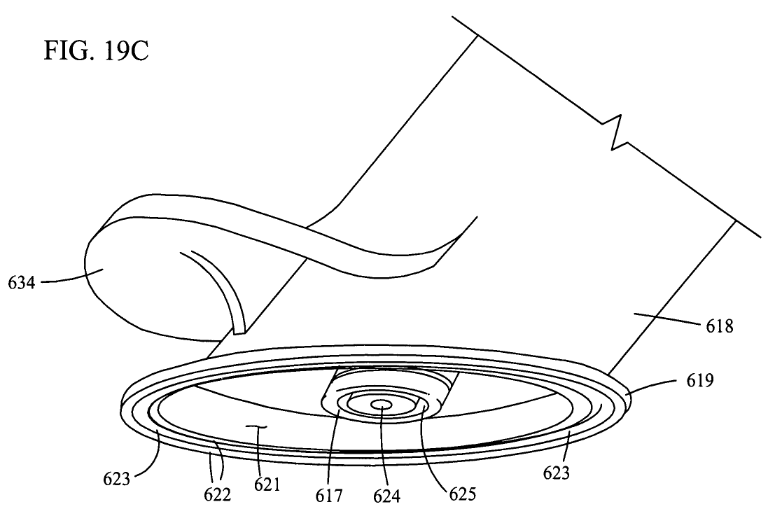

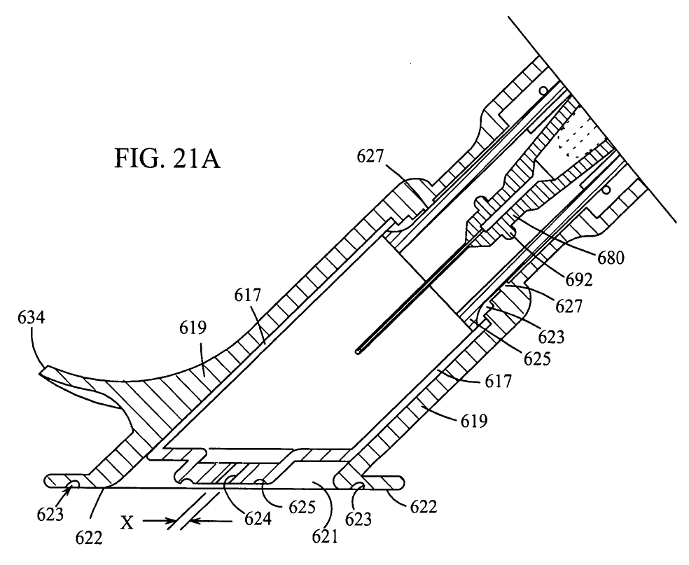

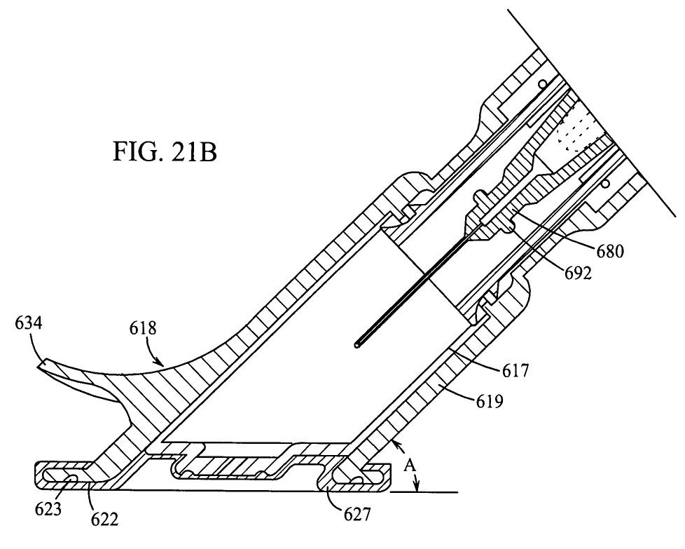

As shown best in FIGS. 19A, 19B, 19C and 21A, the base 618 includes concentric inner and outer shells 617, 619 that define on their underside a radially-extending mounting surface 622 for releasably engaging the skin therebetween, and a needle aperture 624 formed through the approximate center of the inner shell 617 of the base 618. As described in further detail below, and shown in FIG. 21A, a substantially planar needle penetration region “X” is formed on the skin adjacent to the needle aperture 624 upon releasably attaching the base 618 of the device 610 to the skin. The base or barrel assembly 618 of the device 610 includes an annular groove or channel 623 in the outer shell 619 for improving the vacuum seal of the vacuum chamber 621. The base assembly 618 and/or the skin-engaging surfaces thereof may define a non-slip surface for engaging the patient's skin that may be formed, for example, of rubber, Kraton™, PTFE, or any other suitable elastomeric or polymeric material. Preferably, the annular channel 623 contains a sealant, such as a lubricant, gel or the like to improve the seal at the interface between the outer shell 619 and the patient's skin. Similarly, the inner shell 617 also includes an annular groove or channel 625 for receiving a sealant, such as a lubricant, gel or the like, as well. As may be recognized by those of ordinary skill in the pertinent art based on the teachings herein, the sealant may take the form of any of numerous different substances that are currently, or later become known for performing the function of the sealant as described herein, including, for example, a silicon gel, a petroleum jelly, an alcohol-based gel, or a lubricating compound containing an antiseptic, antibacterial and/or anesthetic substance for cleaning or otherwise maintaining the sterility of the contact region of the skin and/or anesthetizing the contact region of the skin.

Referring now to FIG. 21B, a boot 627 for maintaining sterility within the barrel assembly 618 may be provided on the lower end of the device 610. Preferably, the boot 627 is pierceable by the needle. Hence, during use, the boot 627 contacts the skin and maintains the sterility within the barrel assembly 618. In another embodiment (not shown), the boot 627 defines a bore for allowing the needle to pass therethrough. Alternatively, the boot 627 may be manually removed prior to use of the device 610. In one method of assembly, the boot 627 is overmolded onto the barrel assembly 618, although it will be appreciated by those of ordinary skill in the pertinent art that different attachment methods are available.

As described further below, and as shown in FIG. 20, a needle 628 is fixedly secured to one end of the syringe 614 and is movable through the needle aperture 624 upon actuation of the syringe 614 to inject a substance contained within the syringe 614 into the substantially planar needle penetration region X of the skin. In a currently preferred embodiment of the present invention, the needle aperture 624 is sufficiently large to allow the needle 628 to pass therethrough. Otherwise, the diameter or width of the needle aperture 624 may be minimized in order to facilitate maintaining the needle penetration region X of the skin underlying the aperture 624 in a substantially planar condition during injection of the substance contained in the syringe 614 into the skin. In a currently preferred embodiment of the present invention, the needle 628 typically is within the range of a 27 gauge to 30 gauge needle, and the needle aperture 624 defines a diameter or width within the range of about 1 to about 2 mm which, in turn, defines the diameter or width of the needle penetration region X of the skin. As may be recognized by those of ordinary skill in the pertinent art based on the teachings herein, these dimensions are only exemplary, and may be changed as desired depending upon any of numerous different factors.

A releasable backing (not shown) defining a radially-extending peel tab may be releasably secured to the mounting surface 622 of the base and superimposed over the sealant to seal the end of the device and retain the sealant therein during transportation and storage. In the case, immediately prior to use, a user pulls the peel tab away from the base 618 to, in turn, remove the releasable backing and expose the underlying sealant 626. Then, as described further below, the user presses the base onto the skin to releasably secure the mounting surface 622 to the skin by vacuum. The housing 615 further defines a second finger grip 634 axially spaced adjacent to the base 618 to facilitate holding the mounting surface 622 against the skin. The sealant 626 substantially improves the vacuum seal between the skin and the base to thereby define a fixed, substantially planar needle penetration region X on the skin. The ability to form a substantially planar needle penetration region X on the skin is a significant advantage of the device 610 of the present invention because the needle tip can be precisely located within the derma of the skin upon reaching the inward end of the plunger stroke. For example, the device 610 of the present invention may enable the needle tip to be precisely located within a penetration zone of less than about 5 mm in depth, and preferably within the range of about 1 mm to about 3 mm in depth. In addition, the device 610 of the present invention enables such precise locating of the needle tip from one device 610 to the next.

In the illustrated embodiment of the present invention, the mounting surface 622 defines a circular periphery and is tilted at an acute angle “A” (see FIG. 21B) relative to the axis of the device 610. Preferably, the angle A is within the range of about 30° to about 60°, and in the illustrated embodiment is about 45°. However, as may be recognized by those of ordinary skill in the pertinent art based on the teachings herein, the angle A may take different magnitudes to facilitate operation of the device 610. Similarly, the peripheral shape of the mounting surface 622 may take any of numerous different shapes, such as an oval shape, to facilitate releasably securing the skin or otherwise to facilitate the operation of the device 610. In addition, although the illustrated mounting surface 622 is smooth with an inner and outer groove, this surface may take any of numerous different shapes to facilitate engaging the skin or otherwise to facilitate operation of the device 10. The sealant 626 and releasable backing likewise may take the form of any of numerous different types of sealants and/or releasable backings that are currently or later become known for performing the functions of these components of the device 610.

As shown best in FIG. 20, the syringe 614 comprises a hollow syringe body 636 slidably received within the hollow interior 616 of the housing body 615. The syringe body 636 defines a hollow interior forming a chamber 638 therein for receiving the substance to be injected into the skin, a tip 640 formed at one end of the syringe body 636 and defining an aperture 642 therethrough in fluid communication with the substance chamber 638, and a peripheral flange 644 formed at the opposite end of the syringe body 636. In the currently preferred embodiment, the syringe body 636 is made of glass. However, as may be recognized by those of ordinary skill in the pertinent art based on the teachings herein, the syringe body 636 may be made of any of numerous different materials that are currently, or later become known for forming syringes and may take any of numerous different shapes or configurations.

Still referring to FIG. 20, a plunger assembly 646 of the syringe 614 includes a plunger shaft 647 slidably received within the chamber 638 of the syringe body 636, and a resilient tip 648 on the interior end of the plunger shaft 647 that sealingly engages about its periphery the interior wall of the syringe body 636. As shown in FIG. 20, the plunger tip 648 preferably defines a plurality of raised ribs 650 axially spaced relative to each other for forming a fluid-tight seal between the plunger assembly 646 and syringe body 636 while allowing slidable movement therebetween. If desired, the plunger shaft 647 and plunger tip 648 may take the form of a resealable stopper as disclosed in co-pending U.S. patent application Ser. No. 09/781,846, filed Feb. 12, 2001, entitled “Medicament Vial Having A Heat-Sealable Cap, And Apparatus And Method For Filling The Vial”, and U.S. patent application Ser. No. 10/265,075, filed Oct. 3, 2002, entitled “Syringe And Reconstitution Syringe”, each of which is hereby expressly incorporated by reference as part of the present disclosure.

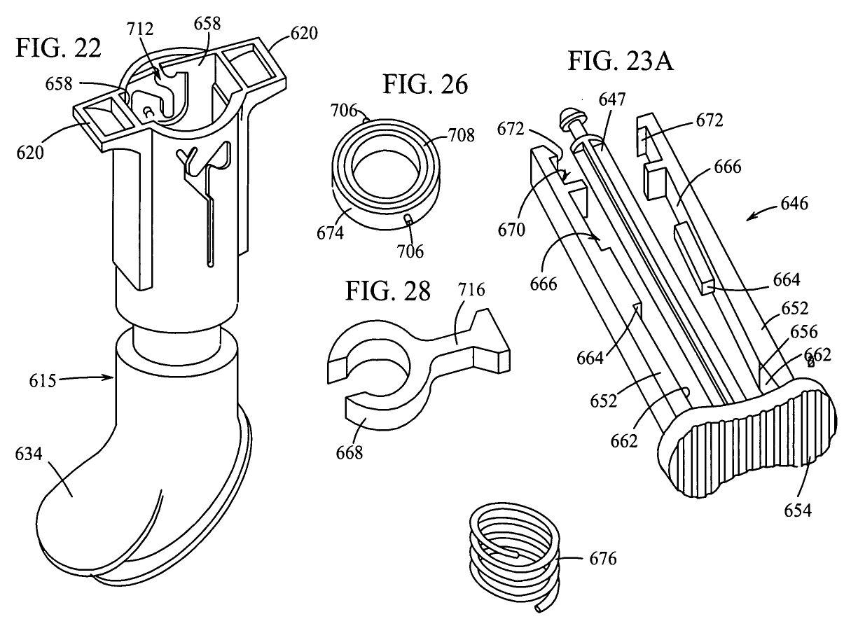

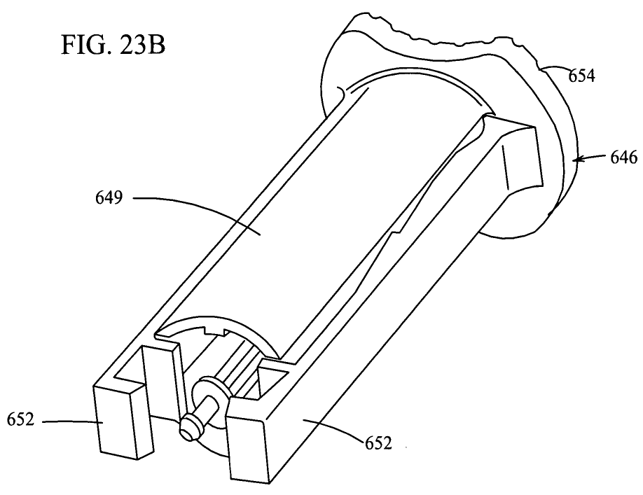

As best seen in FIGS. 23A and 23B, the plunger assembly 646 further includes a pair of diametrically-opposed actuation arms 652 radially spaced relative to the plunger shaft 647 and slidably received within the open end of the housing body 615. As shown best in FIGS. 20 and 22, the housing defines a pair of diametrically-opposed actuation channels 658 for slidably receiving therein the actuation arms 652 of the plunger assembly 646. As shown best in FIG. 20, a shoulder 660 is formed at the base of each actuation channel 658 to stop further movement of the actuation arms and plunger assembly 646. Preferably, a sleeve 649 is included on the plunger assembly 646 to protect tampering with the syringe body prior to use (see FIG. 23B). The outer end of the plunger assembly 646 defines a ribbed surface 654 to facilitate gripping the device 610 by placing a thumb on the ribbed surface 654 and two fingers of the same hand (preferably the index and middle fingers) on each of the first finger grips 620. The user may then place the thumb of the other hand on the second grip 634 to stabilize the device 610 against the skin while simultaneously depressing the plunger assembly 646 by pushing the thumb against the ribbed surface 654 to thereby actuate the device 610. The inward stroke of the plunger assembly 646 drives the syringe body 636 inwardly and, in turn, creates a vacuum adhesion to the skin and drives the needle 628 through the needle aperture 624 and into the derma. The device 610 injects the substance contained in the chamber 638 in a manner similar to that described above in greater detail and, for simplicity, not further described again.

Each actuation arm 652 of the plunger assembly 646 defines a cam surface 656 that tapers inwardly in the direction from the outer to the inner end of the plunger assembly. As can be seen in FIG. 20, each cam surface 656 slidably engages the peripheral flange 644 of the syringe body 636 upon pressing the plunger assembly 646 into the housing body 615. As described further below, the taper of each cam surface 656 allows the plunger shaft 647 to slidably move relative to and within the syringe body 636, while simultaneously maintaining a downward pressure on the syringe body 636 to, in turn, drive the needle 628 through the needle aperture 628 and into the penetration region X of the skin.

Each actuation arm 652 defines a radially-expanded region 662 formed at the juncture of each arm 652 and the gripping portion 654 for capturing therein the peripheral flange 644 of the syringe body 636 upon reaching the end of the plunger stroke. Each actuation arm 652 also defines a first shoulder 664 formed at the inner end of each tapered cam surface 656 for engaging the underside of the peripheral flange 644 of the syringe body 636 and preventing further outward movement of the plunger assembly 646. Each actuation arm 652 further defines a first recess 666 axially spaced relative to the first shoulder 664 for receiving therein a locking ring 668 to prevent inadvertent or other unwanted actuation of the syringe 614. A second recess 670 and second shoulder 672 are formed at the inner end of each actuation arm 652 for capturing therein a rotatable track follower 674. A coil spring 676 is seated within the housing body 615 between a plurality of angularly spaced spring mounts 678 formed within the housing body 615 and the track follower 674, for biasing the plunger assembly 646 outwardly and, in turn, allowing for automatic withdrawal of the plunger assembly 646 and needle 628 from the skin upon injecting the substance therein.

As shown best in FIG. 20, a needle mount 680 is mounted over the inner end 641 of the syringe body 636 and defines on one end a peripheral flange 682 and an elongated aperture 684 formed therethrough. The needle 628 is fixedly secured to the free end of the needle mount 680 and is coupled in fluid communication with the aperture 684 and syringe chamber 638. As also shown in FIG. 20, the peripheral flange 682 of the needle mount is slidably mounted against the proximal end of the contact seal 625 to allow reciprocal movement of the syringe 614 and needle 628 within the housing body 615 therewith. A stop 688 is formed at the base of the outer shell and is engageable with the sealing flange 625 of the contact seal to thereby define the inner end of the plunger/needle stroke. As can be seen, the axial distance between the peripheral stop 688 and the contact seal may be precisely controlled to thereby precisely control the depth of needle penetration into the skin. As may be recognized by those of ordinary skill in the pertinent art based on the teachings herein, the stop for controlling the penetration depth of the needle can be defined by any of numerous different surfaces or other structures that are currently or later become known for performing this function. For example, if desired, the stop can be alternatively defined by a peripheral flange (not shown) formed on the proximal end of the contact seal 625 that is engageable with a corresponding flange (not shown) formed on the body 618, such as the flange 678. The stop feature, in combination with the substantially planar needle penetration region X of the skin formed by the vacuum tensioning of the skin across the needle aperture, enables reliable and precise penetration of the needle tip into the derma.

As shown best in FIG. 21A, the peripheral edge of the contact seal 625 slidably engages the inner wall of the inner shell 617 and forms a gas-tight seal therebetween for creating a vacuum within the vacuum chamber 621 and variable volume chamber 623 upon sliding the contact seal axially outwardly toward the distal end of the device. The body 618 defines an annular flange 627 formed in the inner surface of the body and spaced axially inwardly relative to the vacuum port(s) 624. The annular flange 627 slidably engages the exterior surface of the axially-elongated body portion of the contact seal 625 to form a gas-tight seal therebetween, and thereby enable the creation of a vacuum within the variable volume chamber 623 and vacuum chamber 621 with axial movement of the contact seal. Preferably, the contact seal, body and inner shell are formed of suitable polymeric materials that facilitate formation of the gas-tight seals between the sliding parts. One advantage of the illustrated embodiment, is that the plastic-on-plastic seals obviate the need for an additional o-ring or other gasket to hermetically seal the vacuum chamber.

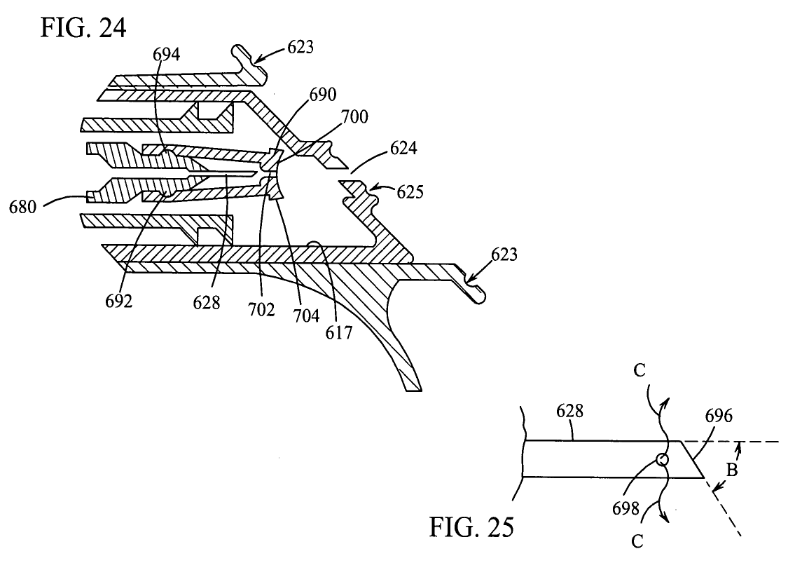

As shown in FIG. 24, a needle cap 690 is mounted over the end of the needle mount 680 to seal the needle 628 and syringe 614 during filling and storage. The needle mount 680 defines an annular rib 692 and the needle cap 690 defines a corresponding annular recess 694 for receiving therein the rib 692 and fixedly securing the needle cap 690 to the needle mount 680. Preferably, the interface between the needle 628, cap 690 and needle mount 680 defines a fluid tight or hermetic seal to maintain the sterility of the needle and of the substance contained within the syringe 614.

As shown in FIGS. 24 and 25, the needle 628 is preferably a “non-coring” needle defining a closed end surface or tip 696 and at least one, and preferably two apertures 698 located adjacent to the closed tip 696. In the illustrated embodiment, the apertures 698 are located on diametrically opposite sides of the needle relative to each other. However, as may be recognized by those of ordinary skill in the pertinent art based on the teachings herein, each needle aperture 698 may take any of numerous different shapes and/or configurations, and the needle 628 may include one or more of such apertures at different desired locations. Each needle aperture 698 is coupled in fluid communication with the syringe chamber 638 and, as indicated by the arrows “C” in FIG. 25, the fluid or other substance contained within the syringe chamber 638 flows laterally outwardly through the apertures 698 and into the derma upon penetration of the needle tip therein.

As shown in FIG. 25, the closed end surface or tip 696 of the needle 628 is oriented at an acute angle “B” relative to the axis of the device 610. Preferably, the angle B is approximately equal to the angle A of the base surface 622 shown in FIG. 21B to facilitate penetration of the needle tip to a precise, predetermined depth into the skin and, in turn, facilitate efficient and effective injection of the substance of the syringe 614 into the derma of the skin. However, as may be recognized by those of ordinary skill in the pertinent art based on the teachings herein, the angle “B” may be set as required to facilitate effective operation of the device 610. A significant advantage of the non-coring needle 628 of the device 610 of the present invention is that the non-coring needle 628 facilitates in reducing the head loss that otherwise might be created by the occlusion of tissue cells that can occur in typical prior art needles. Such head loss undesirably increases the pressure required to depress the plunger assembly 646 which results in a correspondingly higher pressure of the substance released from the syringe 614. It is believe that the release pressure could, in some cases, be excessive to the point where the substance injected might undesirably perforate the basal membrane of the derma. This potential problem is further alleviated by the inclusion of multiple release outlets in the non-coring needle which results in a correspondingly lower release pressure. Yet another advantage of the non-coring needle 628 of the device 610 of the present invention is that the substance injected through the needle apertures 698 flows generally laterally through the derma, rather than perpendicular to, inwardly or under the derma of the skin. Thus, the injected substance does not need to perforate the cells but just disconnect the adhesiveness of the cells and insinuate on the sides of the non-coring needle.

As shown in FIG. 24, the needle cap 690 defines a closed end 700 forming a normally-closed aperture 702 forming a needle guide for receiving therein the tip of the needle 628. The closed end 700 of the needle cap 690 defines a peripheral flange 704 having a diameter or width greater than the diameter or width of the needle aperture 624 formed through the base of the inner shell 617. Accordingly, on the inward stroke of the plunger assembly 646 and needle 628, the peripheral flange 704 of the needle cap 690 engages the base surface of the inner shell 617 surrounding the needle aperture 624 to thereby prevent further inward movement of the needle cap 690. Then, the needle 628 continues to move inwardly through the needle guide 703 and pierces the end surface 700 of the needle cap 690 prior to passage through the needle aperture 624 and into the patient's skin. In one embodiment of the present invention, the needle cap 690 is made of an elastomeric material to facilitate forming a fluid-tight or hermetic seal between the needle cap 690 and needle mount 680. This type of material also facilitates the ability of the needle cap to axially compress upon the surface engages the base of the inner shell and the needle passes therethrough. In addition, as shown in FIG. 24, the tip of the needle cap 690 located within the injection path of the needle 628 is made relatively thin to facilitate ease of insertion of the needle tip therethrough. The needle cap 690 may be formed of rubber, Kraton™, PTFE, or any other suitable elastomeric or polymeric material. However, as may be recognized by those of ordinary skill in the pertinent art based on the teachings herein, the needle cap 690 may be made of any of numerous different materials that are currently or later become known for performing the function of the needle cap 690 disclosed herein.

As shown in FIG. 26, the track follower 674 is ring-shaped and defines a pair of diametrically-opposed followers or pins 706 projecting outwardly from the side wall thereof. The track follower 674 further defines a first raised annular bearing surface 708 formed on the outer end thereof for rotatably and slidably contacting the adjacent surface of the cam arms 652, and a second raised annular bearing surface formed on the other end thereof for rotatably and slidably contacting the second shoulder 672 of each cam arm 652. As best seen from FIGS. 23A, 23B, 26 and 27, each track pin 706 is received within a respective slot 712 formed on the side of the housing body 615.

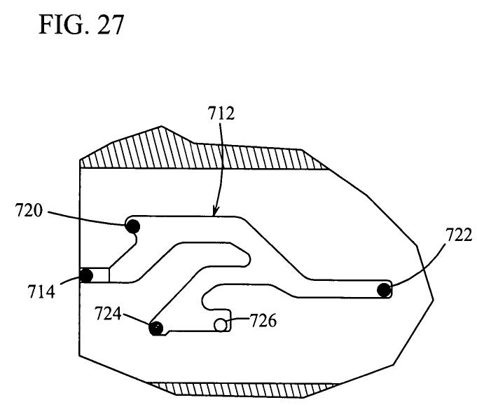

As shown in FIG. 22, the slots 712 are located on opposite sides of the housing body 615 relative to each other, and each slot defines a plurality of track pin positions for controlling actuation of the device 610. As shown in FIG. 27, each slot 712 defines a first pin position 714 defining the entry point for the respective pin 706 into the slot, e.g., the storage position. When located in the first pin position 714, the locking ring 668 is releasably secured to the syringe body 636 and received within the recess 666 (FIGS. 18-20). As shown in FIGS. 18, 19A-C and 28, the locking ring 668 defines a radially-projecting tab 716 and an opening 718 extending through the locking ring 668. As can be seen, the locking ring 668 prevents inward movement of the plunger assembly 646 by means of the tab 716 abutting against the outer end of the housing body 615. Prior to use, a user pulls the tab 716 radially outwardly to thereby release the locking ring 668 from the syringe body 618 and allow actuation of the syringe 614. As may be recognized by those of ordinary skill in the pertinent art based on the teachings herein, the locking ring 668 or like locking device may take any of numerous different shapes and/or configurations to prevent actuation of the device 610 prior to its intended use. For example, the locking ring 668 may be formed of a frangible member that must be broken to remove it from the device 610 and thereby further prevent reuse of the device 610 or components thereof.

Referring again to FIG. 27, each slot 712 defines a second pin position 720 spaced axially inwardly and to the side of the first pin position 714, e.g., the mounting position. The locking ring 668 permits sufficient relative movement of the plunger assembly 646 and syringe body 636 to move the track follower 674 from the first pin position 714 into the second pin position 720. As can be seen in FIG. 27, the angled surfaces of the slots 712 cause the pins 706 to rotate with inward movement of the plunger assembly 646, and the outward pressure applied by the spring 676 (FIGS. 20 and 30) pushes the pins 706 into the second pin position 720 once located within the respective portion of the slot 712. When located in the second pin position 720, the device 610 is ready for use and cannot be disassembled. The third pin position 722 of each slot 712 is spaced axially inwardly and angularly relative to the second pin position 720, and defines the point at which the plunger assembly 646 is fully depressed and the injection completed, e.g., the injecting position.

In order to actuate the device 610 and move the plunger assembly 646 from the second pin position 720 to the third pin position 622, the user must first remove the locking ring 668 by pulling outwardly on the tab 716. Upon completing the injection, the user releases the plunger assembly 646, and the spring 676 is allowed to drive the plunger assembly 646 outwardly until the track follower 674 and pins 706 are received within a fourth pin position 724, e.g., the retracted position. As shown in FIG. 27, each slot 712 defines a fifth pin position 726 axially spaced adjacent to the fourth pin position 724, e.g., the safety position. When located in the fourth pin position 724, any further attempts to actuate the device 610 will result in limited travel between the fourth and fifth pin positions, 724 and 726, respectively, and thus will prevent further actuation and/or use of the device 610. Accordingly, subsequently handling of the device 610 is safe in that the needle tip is not exposed as a potentially contaminated sharp biohazard.

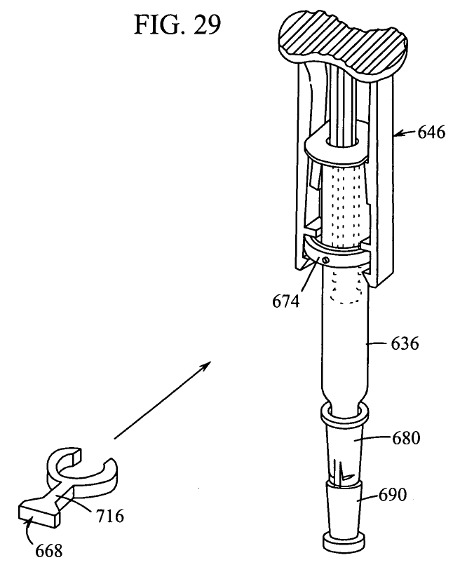

In order to assemble and fill the device 610 of the present invention, the empty syringe bodies 636 are assembled to the needle mounts 680 having the needles 628 fixedly mounted therein. Each needle mount 680 may be press fit onto the end of the respective syringe body 636, or if desired, an epoxy or other suitable bonding material may be applied to the interface to fixedly secure the needle mount 680 to the syringe body 636. As shown typically in FIG. 29, the needle caps 690 are fixed to the needle mounts 680 and the plunger assemblies 646 (including the track followers 674, but not the locking rings 668) are slidably mounted within the syringe bodies 636. Then, each subassembly including the syringe body 636, needle mount 680, needle cap 690 and plunger assembly 746 is sterilized, such as by the application of gamma radiation thereto. However, as may be recognized by those of ordinary skill in the pertinent art based on the teachings herein, any of numerous different methods that are currently or may later become known may be employed to sterilize the components of the device 610 before and/or after filling with the substances to be contained therein. A significant advantage of the needle cap 690 of the device 610 of the present invention is that it allows the syringes 614 to be sterilized and pre-filled prior to assembling the syringes 614 into the housing body 615. Thus, the device 610 of the present invention can be filled with the same type of equipment used to fill prior art syringes. Yet another advantage of the pierceable needle cap 690 of the device 610 of the present invention is that it is contained within the housing body 615, and therefore allows the needle aperture 698 in the housing body 615 to be small enough to permit passage of the needle 628 only therethrough. The relatively small needle aperture facilitates the formation of a substantially planar needle penetration region X on the skin and, in turn, facilitates efficient and effective intradermal delivery.

The sterilized subassemblies are then seated within a filling fixture, such as a tray defining a plurality of recesses or other mounting surfaces for holding a plurality of such subassemblies and transporting them within any of numerous different types of sterile filling machines known to those of ordinary skill in the pertinent. For example, such sterile filling machine may take the form of the filling machine disclosed in U.S. Pat. No. 5,641,004 to Py, entitled “Process For Filling A Sealed Receptacle Under Aseptic Conditions”, and which is hereby expressly incorporated by reference as part of the present disclosure. In addition, and particularly if the plunger shaft 647 and plunger tip 648 take the form of a resealable stopper as described above, the sterile filling machine may take the form of the filling machine disclosed in co-pending U.S. patent application Ser. No. 09/781,846, filed Feb. 12, 2001, entitled “Medicament Vial Having A Heat-Sealable Cap, And Apparatus And Method For Filling The Vial”, incorporated by reference above, or in the U.S. patent application entitled “Sterile Filling Machine Having Needle Filling Station Within E-Beam Chamber”, filed Jun. 19, 2003, under Attorney Docket No. 488180.0094, and which claims priority on U.S. Provisional Patent Application No. 60/390,212, entitled “Sterile Filling Machine Having Needle Filling Station Within E-Beam Chamber”, filed Jun. 19, 2002, each of which is assigned to the Assignee of the present invention and is hereby expressly incorporated by reference as part of the present disclosure.

Upon filling each syringe body 636, the plunger assembly 614 is preferably vacuum capped to the syringe body 636 in a manner known to those of ordinary skill in the pertinent art to form a substantially airless interior within the syringe body 636. As described above, the interface between the plunger 648 and syringe body 636, and the interface between the needle cap 690 and needle mount 680 define substantially airtight or hermetic seals to maintain the airless condition of the substance within the syringe body 636 throughout its shelf life. The filled, airless syringe subassemblies 614 are then mounted within the housing bodies 615 with the springs 676 mounted between the track followers 674 and the spring mounts 678, and the locking rings 668 secured to the syringe bodies 636.

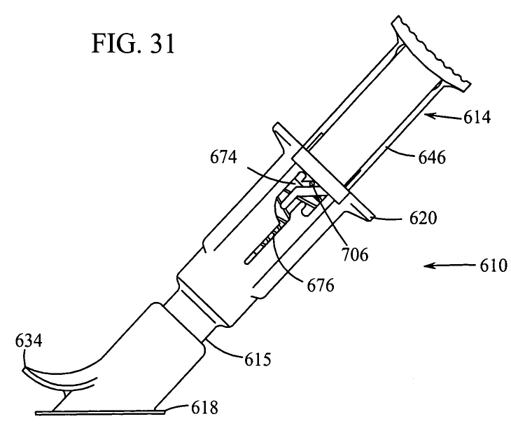

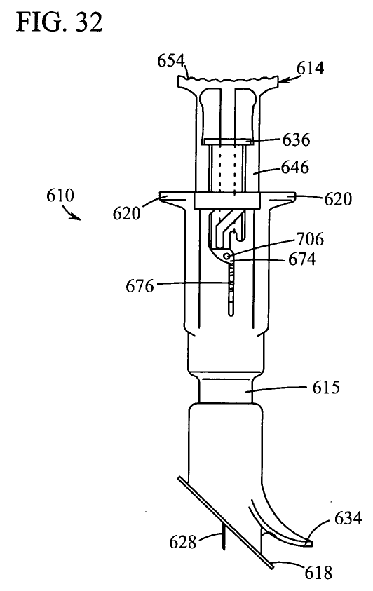

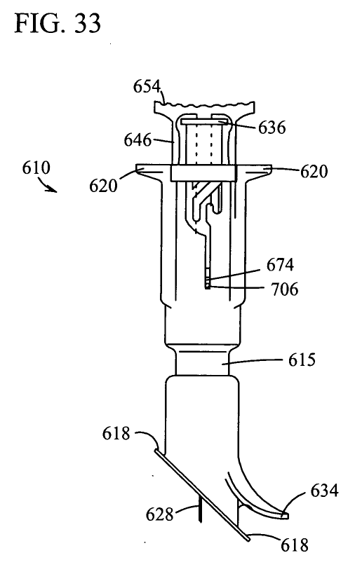

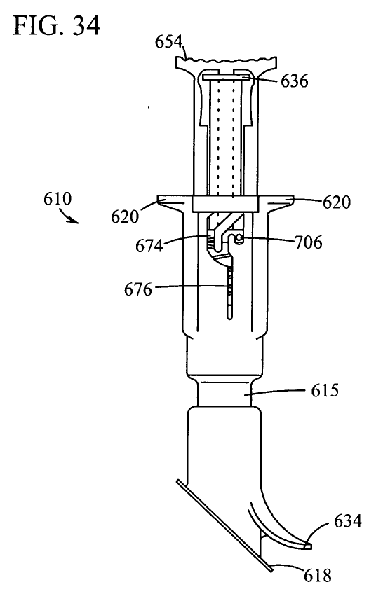

In order to use the device 610 and as shown in FIG. 31, the user removes the locking ring 668 to allow the device 610 to be actuated, and removes the foil or like releasable backing (not shown) from the base 618 of the device 610 to expose the underlying sealant 626 and needle aperture 624. Then, with reference to FIG. 32, the user places the inner and outer shells against the desired portion of the patient's skin and lightly presses the base 618 against the skin by applying the thumb of the other hand to the finger grip 634. The user then applies the index and middle fingers of the other hand to the finger grips 620, and applies the thumb of the same hand to the gripping portion 654 of the plunger assembly 646. Then, the user presses the plunger assembly 646 inwardly using the thumb, index and middle fingers in a “trigger-like” action to, in turn, cause the track follower 674 to compress the coil spring 676 and simultaneously cause the cam surfaces 656 of the actuation arms 652 to engage the peripheral flange 644 of the syringe body 636 and move the syringe body 636 inwardly. Prior to full compression of the spring 676, the contact seal 625 moves axially within the inner shell 617 and, in turn, creates a vacuum between the vacuum chamber 621 and the underlying skin. This, in turn, causes the device 610 to vacuum attach to the skin and thereby create the substantially planar needle penetration region X on the skin by tensioning the skin and preventing relative movement of the skin and device 610. Also prior to full compression of the spring 676, the plunger assembly 646 moves inwardly with the syringe body 636 and thus does not cause the plunger 648 to displace any substance from the syringe 614. At full compression of the spring 676, the track follower 674 and pins 706 are located in the third pin position 722 of FIG. 27. At this point, the needle tip 696 has pierced the end surface 700 of the needle cap 690 and is inserted at a predetermined depth into the needle penetration region X of the skin. Then, as the user continues to press inwardly on the gripping surface 654 of the plunger assembly 646, the plunger tip 648 moves through the syringe chamber 638 to dispense the substance contained therein through the needle holes 698 and into the skin. The actuation arms 652 of the plunger assembly 646 are sufficiently flexible to move over the flange 644 of the syringe body 636 to allow further actuation of the syringe 614. When the plunger tip 648 reaches the inner end or bottom of its stroke, the user releases the thumb from the gripping surface 654 of the plunger assembly 646. At this point, and as shown in FIG. 33, the flange 644 of the syringe body 636 is captured within the recessed portions 666 of the actuation arms 652, and the spring 676 is then allowed to drive the plunger assembly 646 and needle assembly 628 outwardly from the patient's skin. This, in turn, brings with it the contact seal which releases the vacuum upon the skin. The user may then simply lift the device 610 away from the skin. As shown in FIG. 34, the spring 676 drives the track follower 674 and pins 706 into the fourth pin position 724 of FIG. 27 to thereby prevent further actuation of the device 610.

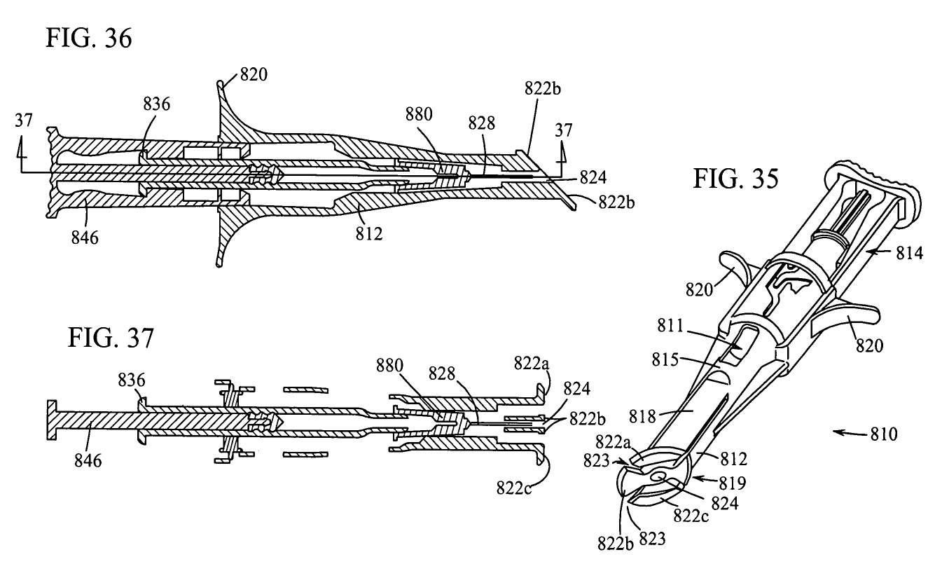

Referring now to FIGS. 35-38, another embodiment of a device that is configured for intradermally delivery is indicated to generally by the reference numeral 810. As will be appreciated by those of ordinary skill in the pertinent art, the device 810 is similar in many respects to the device 610 described above. Accordingly, like reference numerals preceded by the numeral “8” instead of the numeral “6”, are used to indicate like elements. In addition, the description herein is largely directed to the differences for simplicity. The device 810 comprises a housing 812 and a syringe 814 mounted within the housing 812. The housing 812 includes an axially-elongated housing body 815 with a base 818 formed at a lower end 819 of the housing 812. The base 818 includes on its underside an expandable mounting surface 822 defined by a plurality of discrete mounting surfaces 822a-c for tensioning the skin across the needle penetration region X. Preferably, the discrete mounting surfaces 822a-c are a non-slip surface, such as an elastomeric or polymeric coated surface, to engage the patient's skin. However, as may be recognized by those of ordinary skill in the pertinent art based on the teachings herein, the mounting surfaces 822a-c can take any of numerous different configurations to perform the function of engaging the skin as described herein. For example, each surface could be formed with a relatively rough surface finish to facilitate releasably engaging the skin, or each surface could be coated with a substance to facilitate releasable engagement of each such surface with the skin. Expansion slots 823 formed in the lower end 819 of the housing allow mounting surfaces 822a, 822c to expand radially outward; however, mounting surface 822b remains fixed and stable to define the needle aperture 824 through which the needle tip extends. As best seen in FIG. 35, the housing 812 of the device 810 defines a window 811 for inspecting the syringe sub-assembly 814. Accordingly, if tampering is determined by viewing the internal components via the inspection window, the device 810 can be discarded.

When the mounting surfaces 822a, 822c are placed against the patient's skin and the mounting surfaces 822a, 822c expand radially outward, the skin of the needle penetration region X is stretched across the needle aperture 824. The ability to form a taut substantially planar needle penetration region X on the skin is a significant advantage of the device 810 because the natural looseness of the skin has been decreased. As a result, when the needle tip penetrates the derma of the skin, the flatness of the needle penetration region X is substantially maintained to allow accurate prediction of the insertion depth of the needle 828.

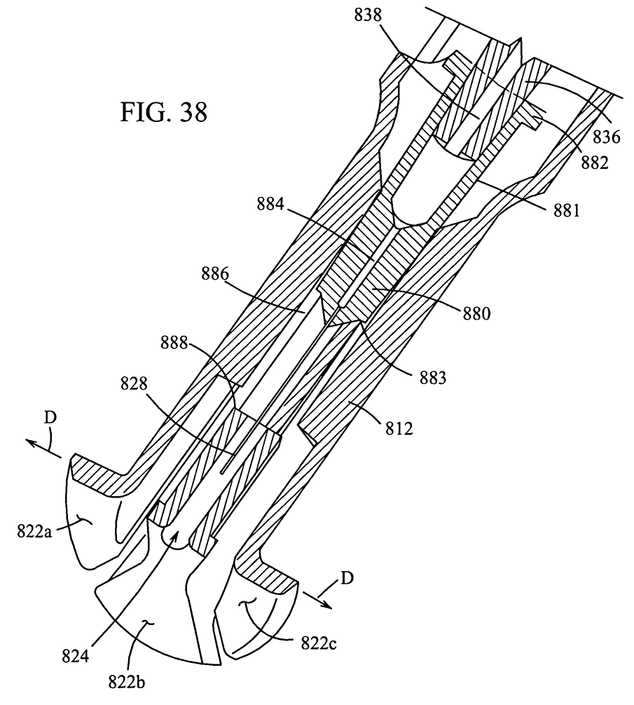

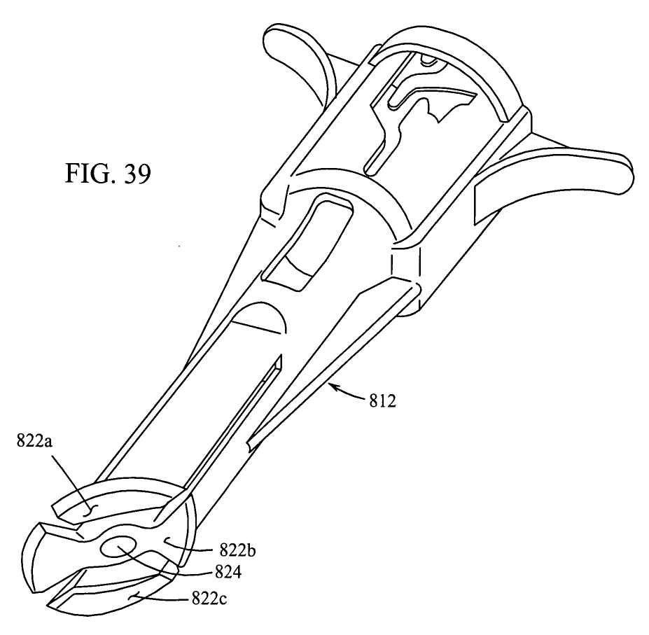

As shown best in FIG. 38, a needle mount 880 is mounted over the inner end 840 of the syringe body 836 and defines on one end a peripheral flange 882 and an elongated aperture 884 formed therethrough. Outer walls 881 of the needle mount 880 are tapered for increasing interference with the housing 812 as the needle mount 880 travels toward the needle aperture 824. The resulting interference causes expansion of the lower end 819 of the housing 812 and, thereby, the mounting surfaces 822a, 822c expand radially outward as indicated by arrows “D”. The needle 828 is fixedly secured to the free end of the needle mount 880 and is coupled in fluid communication with the syringe chamber 838. The needle mount 880 forms a peripheral flange 882 at an upper end and a peripheral shoulder 883 at a lower end. The needle mount 880 is slidably mounted within a bore 886 of the housing 812 to allow reciprocal movement of the syringe 814 and needle 828 within the housing 812. A peripheral stop 888 is formed at the one end of the bore 886 and is engageable with the shoulder 883 of the needle mount 880 to thereby define the inner end of the plunger/needle stroke. As can be seen, the axial distance between the shoulder 883 of the needle mount 880 and the peripheral stop 888 of the housing 812 may be precisely controlled to thereby precisely control the depth of needle 828 penetration into the skin without a practiced skill level on the part of the user.

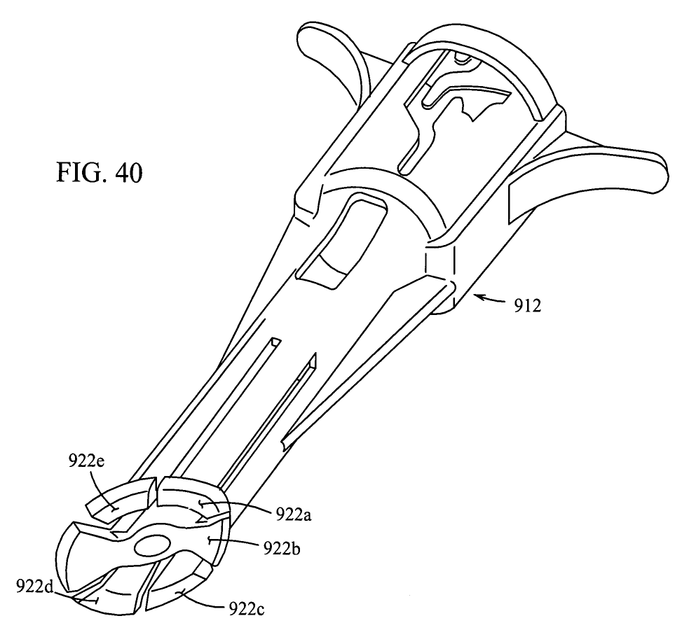

It will be recognized by those of ordinary skill in the pertinent art based upon review of the subject disclosure that many variations are possible. For example, the principles and devices herein can be advantageously used to inject substances other than intradermally, such as sub-cutaneously. Similarly, the devices can be made of any of numerous different materials that are currently, or later become known for performing the functions of the various components of the devices described or otherwise disclosed herein. If desired, the devices may include more than one needle for simultaneously injecting the substance with a plurality of needles into the substantially planar or other target penetration region of the skin. If desired, the multiple needles may be formed, for example, of a plastic material, and injection molded as a needle head on the syringe. In addition, the vacuum chamber and/or the mechanism for creating the vacuum within the vacuum chamber can take any of numerous different configurations that are currently, or later become known for performing this function. Further, the stop surface or surfaces for controlling and/or setting the insertion depth of the needle can take any of numerous different shapes and/or configurations that are currently or later become known for performing this function. For another example, with respect to the device 910 of FIG. 40, the number of discrete mounting surfaces may take a multitude of different configurations wherein a base 918 of a housing 912 may form five mounting surfaces 922a-e. Moreover, the mounting surface may use expandable portions in combination with additional means for tensioning the skin such as vacuum.

Accordingly, this detailed description of preferred embodiments is to be taken in an illustrative, as opposed to a limiting sense.

What is claimed is:

1. An intradermal delivery device, comprising:
a housing including a base defining a needle aperture and a skin-engaging surface extending about a periphery of the needle aperture;
a syringe including a syringe body coupled to the housing and a plunger slidably received within the syringe body;
a needle coupled in fluid communication with the syringe body and movable through the needle aperture to penetrate the skin and inject a substance contained within the syringe body therein;
a vacuum chamber coupled in fluid communication with the base for drawing a vacuum within the base and, in turn, releasably securing the skin-engaging surface to the skin and forming a substantially planar needle penetration region on the skin;
at least one stop surface fixed relative to at least a portion of the skin-engaging surface to define a predetermined distance therebetween, and adapted to cooperate with the needle to limit a depth of insertion of the needle into the needle penetration region of the skin, wherein the needle is movable through the needle aperture upon slidably moving the plunger through the syringe body to thereby penetrate with the needle the penetration region of the skin and inject a substance contained within the syringe body therein.

2. An intradermal delivery device as defined in claim 1, wherein the needle is non-coring and defines least one opening in a side wall thereof.

3. An intradermal delivery device as defined in claim 2, wherein the non-coring needle includes at least two side openings located in approximately opposite sides of the needle relative to each other.

4. An intradermal delivery device as defined in claim 2, wherein the at least one stop surface locates the at least one needle opening at a predetermined depth with the opening located substantially entirely within the derm.

5. An intradermal delivery device as defined in claim 1, wherein the skin-engaging surface is oriented at an acute angle relative to an axis of the intradermal delivery device.

6. An intradermal delivery device as defined in claim 5, wherein the acute angle is within the range of about 30° to about 60° relative to the axis of the intradermal delivery device.

7. An intradermal delivery device as defined in claim 5, wherein the acute angle is about 45° relative to the axis of the intradermal delivery device.

8. An intradermal delivery devise as defined in claim 1, wherein the syringe body is slidably mounted within the housing to move the needle between retracted and skin-penetrating positions.

9. An intradermal delivery as defined in claim 1, wherein the housing further defines a rest axially spaced adjacent to the base for receiving a digit of a user for stabilizing the base against the skin.

10. An intradermal delivery device as defined in claim 1, wherein the syringe is axially movable within the housing, and the at least one stop surface is positioned to engage the syringe and prevent further axial movement of the syringe.

11. An intradermal delivery device as defined in claim 1, further comprising a needle cap mounted over the needle and forming an approximately airtight seal therebetween, and defining a penetrable surface formed adjacent to the needle tip for passage of the needle therethrough.

12. An intradermal delivery device as defined in claim 11, wherein the syringe, needle and needle cap form a sealed, pre-fillable subassembly insertable into the housing after filling the syringe body with a substance.

13. An intradermal delivery device as defined in claim 11, wherein the needle is a non-coring needle defining a closed end surface and at least one aperture located in a side wall thereof in fluid communication with the syringe body.

14. An intradermal delivery device as defined in claim 1, further comprising a removable locking member attachable to the syringe for preventing actuation of the intradermal delivery device prior to removal thereof.

15. An intradermal delivery device as defined in claim 1, further comprising a pair of first and second finger grips formed on the housing on approximately opposite sides of the plunger relative to each other for receiving digits of a first hand, and a third finger grip formed on the housing adjacent to the base for receiving a digit of a second hand for controlling application of the intradermal delivery device to the skin.

16. An intradermal delivery device as defined in claim 15, wherein the first and second finger grips are positioned for receiving an index and middle finger, respectively, and the plunger defines a surface for receiving a thumb of the first hand.

17. An intradermal delivery device as defined in claim 1, wherein the base defines at least one aperture formed adjacent to the skin-engaging surface and coupled in fluid communication with the vacuum chamber for drawing a vacuum through the at least one aperture and releasably securing the skin-engaging surface to the skin.

18. An intradermal delivery device as defined in claim 17, wherein the at least one aperture extends adjacent to a periphery of the skin-engaging surface.

19. An intradermal delivery device as defined in claim 17, wherein the base defines at least one first recess spaced on an opposite side of the vacuum aperture relative to the needle aperture and adapted to receive therein a sealant to facilitate the formation of a vacuum within the vacuum aperture and releasably securing the skin-engaging surface to the skin.

20. An intradermal delivery device as defined in claim 19, wherein the at least one first recess defines a first approximately annular groove.

21. An intradermal delivery device as defined in claim 19, wherein the base further defines at least one second recess located between the needle aperture and the vacuum aperture for receiving therein a sealant to facilitate the formation of a vacuum within the vacuum aperture and releasably securing the skin-engaging surface to the skin.

22. An intradermal delivery device as defined in claim 1, wherein the housing includes first and second parts, wherein at least one of the first and second parts is movable relative to the other to create a vacuum within the vacuum chamber.

23. An intradermal delivery device as defined in claim 22, wherein the syringe body is movable within the housing, and at least one of the first and second parts is movable relative to the other with movement of the syringe body to create a vacuum within the vacuum chamber.

24. An intradermal delivery device as defined in claim 23, wherein the syringe body is movable within the housing upon moving the plunger relative to at least one of the syringe body and housing.

25. An intradermal delivery device as defined in claim 22, wherein the first part includes a peripheral sealing member that slidably contacts the second part and forms a substantially gas-tight seal therebetween to create a vacuum within the vacuum chamber.

26. An intradermal delivery device as defined in claim 25, wherein the peripheral sealing member is defined by a polymeric flange formed on the first part, and the second part defines an axially elongated polymeric surface, and the flange is slidably engageable with the axially-elongated surface to form a substantially gas-tight seal therebetween.

27. An intradermal delivery device as defined in claim 19, wherein the sealant includes at least one of an antibacterial, anti-septic, and anesthetic substance.

28. An intradermal delivery device as defined in claim 1, further comprising a sleeve that extends axially adjacent to the plunger and is spaced radially therefrom for receiving at least a portion of the syringe body therebetween to protect the syringe body in a retracted position.

29. An intradermal delivery device as defined in claim 1, wherein the base defines a first skin-engaging surface formed adjacent to the needle aperture, and a second skin-engaging surface spaced radially outwardly relative to the first skin-engaging surface, and wherein the first skin-engaging surface is spaced axially inwardly relative to the second skin-engaging surface to facilitate radially directed skin movement relative to the first skin-engaging surface and formation of the substantially planar needle penetration region on the skin.

30. A method for intradermal delivery, comprising the following steps:
providing an intradermal delivery device including a housing having a mounting surface and a reciprocally mounted syringe therein;
placing the mounting surface on the skin of a patient;
creating a vacuum between the housing and the skin and, in turn, releasably securing the mounting surface to the skin;
forming a substantially planar target penetration region on the skin;
introducing a needle of the syringe a predetermined depth into the substantially planar target penetration region of the skin; and
injecting a substance from the syringe through the needle into the substantially planar target penetration region of the skin.

31. A method as defined in claim 30, further comprising the step of providing a non-coring needle defining at least one lateral opening in a side wall thereof; introducing the needle into the target penetration region of the skin at a predetermined depth wherein the at least one lateral opening is located substantially entirely within the derm; and injecting the substance laterally through the at least one opening of the needle and into the derm.

32. A method as defined in claim 31, further comprising the step introducing the needle at a predetermined acute angle between the axis of the needle and the substantially planar target penetration region of the skin.

33. A method as defined in claim 31, wherein the step of forming a substantially planar target penetration region on the skin includes tensioning the skin.

34. An intradermal delivery device, comprising:
first means for defining a substantially planar needle penetration region on the skin;
second means for releasably securing by vacuum the first means to the skin and preventing relative movement of the needle penetration region of the skin and the first means;
third means for storing a substance to be injected into the skin;
fourth means for releasing the substance from the third means; and
fifth means in communication with the third means for penetrating the substantially planar needle penetration region of the skin and injecting the substance contained within the third means therein.

35. An intradermal delivery device as defined in claim 34, further comprising a housing including a base defining a skin-engaging surface and a needle aperture formed adjacent thereto, wherein the first means is defined by the skin-engaging surface and a vacuum chamber coupled in fluid communication with the skin-engaging surface for tensioning skin located within the skin-engaging surface and forming the approximately planar needle penetration region thereon.

36. An intradermal delivery device as defined in claim 34, wherein the second means is defined by a vacuum chamber.

37. An intradermal delivery device as defined in claim 34, further comprising a syringe including a syringe body defining a chamber, wherein the third means is defined by the chamber.

38. An intradermal delivery device as defined in claim 34, wherein the fourth means is a plunger slidably received within the third means.

39. An intradermal delivery device as defined in claim 34, wherein the fifth means is a needle.

40. An intradermal delivery device as defined in claim 34, wherein the first skin-engaging surface is approximately annular and extends about a periphery of the needle aperture, and the device further includes a second approximately annular skin-engaging surface radially spaced relative to the first skin-engaging surface, and wherein the vacuum chamber is coupled in fluid communication between the first and second skin-engaging surfaces for forming a vacuum therein.

41. An intradermal delivery device as defined in claim 35, wherein an axis of the housing forms an oblique angle with respect to the substantially planar needle penetration region.

42. An intradermal delivery device as defined in claim 35, wherein the housing forms a groove about the substantially planar needle penetration region for receiving a sealant.

43. An intradermal delivery device as defined in claim 42, wherein the sealant includes at least one of an anti-septic agent, an anti-bacterial agent, an alcohol, and an anesthetic agent.

44. An intradermal delivery device as defined in claim 39, wherein the needle defines a beveled tip, and the device further comprises a needle mount for coupling the needle to the third means, and the needle mount includes a keyed portion for orienting the beveled tip.

45. An intradermal delivery device as defined in claim 34, further comprising means for limiting a depth of insertion of the fifth means into the substantially planar needle penetration region of the skin.

46. An intradermal delivery device, comprising:
a housing including a base defining a needle aperture and a skin-engaging surface extending about a periphery of the needle aperture;
a syringe including a syringe body coupled to the housing and a plunger slidably received within the syringe body;
a needle coupled in fluid communication with the syringe body and movable through the needle aperture to penetrate the skin and inject a substance contained within the syringe body therein;
at least one stop surface fixed relative to at least a portion of the skin-engaging surface to define a predetermined distance therebetween, and adapted to cooperate with the needle to limit a depth of insertion of the needle into the needle penetration region of the skin; and
means for forming a substantially planar needle penetration region on the skin.

47. An intradermal delivery device as defined in claim 46, wherein the means for forming a substantially planar needle penetration region on the skin is defined by at least a portion of the skin-engaging surface that is radially expandable, and wherein the needle is movable through the needle aperture upon slidably moving the plunger through the syringe body to thereby penetrate with the needle the penetration region of the skin and inject a substance contained within the syringe body therein.

48. An intradermal delivery device as defined in claim 46, wherein the means for forming a substantially planar needle penetration region on the skin is defined by a vacuum chamber coupled in fluid communication with the base for drawing a vacuum within the base and, in turn, releasably securing the skin-engaging surface to the skin and forming a substantially planar needle penetration region on the skin.

49. An intradermal delivery device as defined in claim 47, wherein the portion of the skin-engaging surface is radially expandable in response to movement of the plunger through the syringe body.

50. An intradermal delivery device as defined in claim 47, wherein the housing defines expansion slots for facilitating radial expansion of the mounting surface.

51. An intradermal delivery device as defined in claim 47, further comprising a needle mount for coupling the needle to the syringe body, the needle mount having a tapered wall for radially expanding the portion of the skin-engaging surface.

52. An intradermal delivery device as defined in claim 47, wherein an inner portion of the skin-engaging surface defines the needle aperture and is fixed relative to the radially-expandable portion of the mounting surface.
